(19) United States
(12) Patent Application Publication
(10) Pub. No.: US 2003/0162662 A1
(43) Pub. Date: Aug. 28, 2003

(54) 2-Phenyl-2h-pyridazine-3-ones

(76) Inventors: Michael Puhl, Lampertheim (DE); Thorsten Volk, Mannheim (DE); Gerhard Hamprecht, Weinheim (DE); Robert Reinhard, Ludwigshafen (DE); Ingo Sagasser, Dannstadt-Schauernheim (DE); Cyrill Zagar, Ludwigshafen (DE); Matthias Witschel, Bad Durkheim (DE); Helmut Walter, Obrigheim (DE)

Correspondence Address: Keil & Weinkauf, 1101 Connecticut Avenue NW, Washington, DC, 20036, US

(21) Appl. No.: 10/257,329

(22) PCT Filed: Apr. 12, 2001
(86) PCT No.: PCT/EP01/04213

(30) Foreign Application Priority Data
Apr. 14, 2000 (DE) 100 18 632.7

(51) Int. Cl. A01N 43/58; C07D 237/02

(52) U.S. Cl. 504/238; 544/239

(57) ABSTRACT

The invention relates to 2-phenyl-2H-pyridazin-3-ones of the general formula I

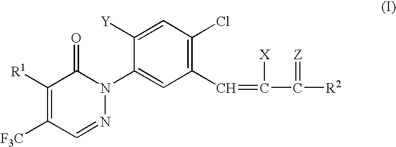

in which the variables R¹, R², X, Y and Z have the following meanings:

X is halogen;

Y is fluorine or chlorine;

Z is oxygen;

R¹ is hydrogen or C₁-C₄-alkyl;

R² is chlorine, OR³ or NR⁴R⁵, in which R³, R⁴ and R⁵ have the meanings indicated in claim 1,

Z can also be a group NR⁶ if R² is a group OR⁷, in which R⁶ and R⁷ have the meanings indicated in claim 1,

and to the agriculturally utilizable salts of compounds of the formula I.

The invention furthermore relates to the use of compounds I and their salts as herbicides and/or for the desiccation and/or defoliation of plants, herbicidal compositions and compositions for the desiccation and/or defoliation of plants, which contain the compounds I and/or their salts as active substances, and processes for controlling undesired vegetation (weeds) and for the desiccation and/or defoliation of plants using the compounds I and/or their salts.

The present invention relates to 2-phenyl-2H-pyridazin-3-ones and to their use as herbicides and/or for the desiccation and/or defoliation of plants.

2H-Pyridazin-3-ones which have a phenyl substituent in the 2-position of the pyridazinone ring are variously described as herbicides in the prior art, for example in WO 96/39392, WO 97/07104, DE 19754348 and WO 99/52878. As a rule, the compounds described there have a substituent, for example a halogen atom, on the phenyl ring in the 2- and/or the 4-position relative to the pyridazinonyl radical. A side chain can be present in the 5-position of the phenyl ring. Inter alia, compounds are proposed which have an ethylenically unsaturated side chain derived from propenoic acid derivatives. The compounds described there have no α-halogen atom in the ethylenically unsaturated side chain.

The compounds of the prior art are frequently not satisfactory with respect to their herbicidal activity and their selectivity. It is therefore an object of the present invention to make available compounds having high herbicidal activity and selectivity.

We have surprisingly found that this object is achieved by certain 5-trifluoromethyl-2H-pyridazin-3-ones which, in the 2-position of the pyridazinone ring, have a phenyl ring which carries a chlorine atom in the 4-position and a further halogen atom in the 2-position and which in the 5-position has a side chain which is derived from an α-halopropenoic acid derivative, and have a high herbicidal activity with simultaneous tolerability for crop plants.

Accordingly, the present invention relates to 2-phenyl-2H-pyridazin-3-ones of the general formula I

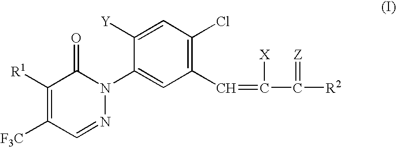

in which the variables R¹, R², X and Z have the following meanings:

X is halogen;

Y is fluorine or chlorine,

Z is oxygen;

R¹ is hydrogen or C₁-C₄-alkyl;

R² is chlorine, OR³ or NR⁴R⁵, in which

R³,R⁴ independently of one another are hydrogen, C₁-C₄-alkyl, C₁-C₄-haloalkyl, hydroxy-C₁-C₄-alkyl, C₁-C₄-alkoxy-C₁-C₄-alkyl, cyano-C₁-C₄-alkyl, C₁-C₄-alkylthio-C₁-C₄-alkyl, C₁-C₄-alkylsulfinyl-C₁-C₄-alkyl, C₁-C₄-alkylsulfonyl-C₁-C₄-alkyl, amino-C₁-C₄-alkyl, C₁-C₄-alkylamino-C₁-C₄-alkyl, di(C₁-C₄-alkyl)amino-C₁-C₄-alkyl, hydroxycarbonyl-C₁-C₄-alkyl, (C₁-C₄-alkoxy)carbonyl-C₁-C₄-alkyl, C₁-C₄-haloalkyloxycarbonyl-C₁-C₄-alkyl, (C₁-C₄-alkylthio)carbonyl-C₁-C₄-alkyl, aminocarbonyl-C₁-C₄-alkyl, (C₁-C₄-alkyl)aminocarbonyl-C₁-C₄-alkyl, di(C₁-C₄-alkyl)aminocarbonyl-C₁-C₄-alkyl, C₁-C₄-haloalkoxy-C₁-C₄-alkyl, C₁-C₄-alkoxy-C₁-C₄-alkoxy-C₁-C₄-alkyl, hydroxycarbonyl-C₁-C₄-alkoxy-C₁-C₄-alkyl, (C₁-C₄-alkoxy)carbonyl-C₁-C₄-alkoxy-C₁-C₄-alkyl, aminocarbonyl-C₁-C₄-alkoxy-C₁-C₄-alkyl, (C₁-C₄-alkyl)aminocarbonyl-C₁-C₄-alkoxy-C₁-C₄-alkyl, di(C₁-C₄-alkyl)aminocarbonyl-C₁-C₄-alkoxy-C₁-C₄-alkyl, C₃-C₆-alkenyl, C₃-C₆-alkynyl, C₃-C₄-alkenyloxy-C₁-C₄-alkyl, C₃-C₄-alkynyloxy-C₁-C₄-alkyl, C₃-C₄-alkenyloxycarbonyl-C₁-C₄-alkyl, C₃-C₄-alkynyloxycarbonyl-C₁-C₄-alkyl, C₃-C₆-cycloalkyl, C₃-C₈-cycloalkyl-C₁-C₄-alkyl, C₃-C₈-cycloalkoxy-C₁-C₄-alkyl; and

R⁵ is hydrogen, C₁-C₄-alkyl, C₁-C₄-alkoxy, C₃-C₆-alkenyl, C₃-C₄-alkenyloxy, C₃-C₆-alkynyl, C₃-C₄-alkynyloxy, C₃-C₈-cycloalkyl, C₃-C₈-cycloalkyl-C₁-C₄-alkyl, C₃-C₈-cycloalkyl-C₁-C₄-alkoxy;

R⁴ and R⁵, together with the nitrogen atom to which they are bonded, can also be a preferably saturated or unsaturated 3-, 4-, 5-, 6- or 7-membered heterocyclic radical which contains 1 or 2 further, preferably nonadjacent, heteroatoms, selected from oxygen and sulfur, and/or one or two imino or C₁-C₄-alkylimino groups as ring members and/or one or two substituents selected from halogen, C₁-C₄-alkyl and C₁-C₄-alkoxy;

Z can also be a group NR⁶ if R² is a group OR⁷, in which

R⁶ is C₁-C₄-alkoxy, C₃-C₄-alkenyloxy, C₃-C₄-alkynyloxy, C₃-C₈-cycloalkoxy, C₃-C₈-cycloalkyl-C₁-C₄-alkoxy; and

R⁷ is C₁-C₄-alkyl, C₁-C₄-haloalkyl, C₁-C₄-alkoxy-C₁-C₄-alkyl, cyano-C₁-C₄-alkyl, C₁-C₄-alkylthio-C₁-C₄-alkyl, hydroxycarbonyl-C₁-C₄-alkyl, (C₁-C₄-alkoxy)carbonyl-C₁-C₄-alkyl, aminocarbonyl-C₁-C₄-alkyl, (C₁-C₄-alkyl)aminocarbonyl-C₁-C₄-alkyl, di(C₁-C₄-alkyl)aminocarbonyl-C₁-C₄-alkyl, C₁-C₄-haloalkoxy-C₁-C₄-alkyl or C₁-C₄-haloalkyloxycarbonyl-C₁-C₄-alkyl, C₁-C₄-alkoxy-C₁-C₄-alkoxy-C₁-C₄-alkyl;

and the agriculturally utilizable salts of compounds of the formula I.

The invention furthermore relates to

the use of compounds I and their salts as herbicides and/or for the desiccation and/or defoliation of plants,

herbicidal compositions and compositions for the desiccation and/or defoliation of plants which contain the compounds I and/or their salts as active substances,

processes for controlling undesired vegetation (weeds) and for the desiccation and/or defoliation of plants using the compounds I and/or their salts.

The invention moreover relates to diazinylcinnamic acid compounds of the general formula II

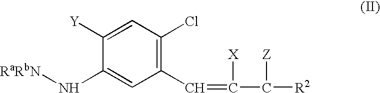

in which R^{a} and R^{b} are simultaneously hydrogen or form a group ═CH—C(O)—CF₃ and the variables R², R³, X, Y and Z have the meanings mentioned beforehand. Among these, preferred compounds II are those in which Z is oxygen and R² is a group OR³, where R³ has the meanings indicated beforehand, preferably other than hydrogen, in particular the meanings indicated below as preferred.

Among the compounds of the formula II, those compounds are particularly preferred in which Z is oxygen and R² is selected from C₁-C₄-alkoxy. The compounds of the formula II are suitable intermediates for the preparation of the compounds of the general formula I according to the invention.

With respect to the double bond in the side chain, the compounds of the formula I can be present as E or Z isomers, based on the relative arrangement of phenyl ring and halogen atom X. The compounds I according to the invention include both the pure E or Z isomers, and also their mixtures. The Z isomer is preferred, both in pure form and in the form of mixtures which contain the Z isomer in enriched form.

The compounds of the formula I can have one or more chiral centers in the substituents and then exist either as enantiomer or diastereomer mixtures. The invention relates both to the pure enantiomers or diastereomers and to their mixtures.

Suitable agriculturally utilizable salts are especially the salts of those cations or the acid addition salts of those acids whose cations or anions respectively do not adversely affect the herbicidal action of the compounds I. Thus suitable cations are in particular the ions of the alkali metals, preferably sodium and potassium, the alkaline earth metals, preferably calcium, magnesium and barium, and the transition metals, preferably manganese, copper, zinc and iron, and also the ammonium ion, which if desired can carry one to four C₁-C₄-alkyl substituents and/or a phenyl or benzyl substituent, preferably diisopropylammonium, tetramethylammonium, tetrabutylammonium, trimethylbenzylammonium, in addition phosphonium ions, sulfonium ions, preferably tri(C₁-C₄-alkyl)sulfonium and sulfoxonium ions, preferably tri(C₁-C₄-alkyl)sulfoxonium.

Anions of utilizable acid addition salts are primarily chloride, bromide, fluoride, hydrogensulfate, sulfate, dihydrogenphosphate, hydrogenphosphate, phosphate, nitrate, hydrogencarbonate, carbonate, hexafluorosilicate, hexafluorophosphate, benzoate, and the anions of C₁-C₄-alkanoic acids, preferably formate, acetate, propionate and butyrate. They can be formed by reaction of I with an acid of the corresponding anion, preferably of hydrochloric acid, hydrobromic acid, sulfuric acid, phosphoric acid or nitric acid.

The organic moieties mentioned in the definition of the substituents R¹-R⁷ or as radicals on cycloalkyl rings or on Z—such as the meaning halogen—are collective terms for individual lists of the separate group members. All carbon chains, i.e. all alkyl, haloalkyl, cyanoalkyl, hydroxyalkyl, aminoalkyl, cycloalkylalkyl, alkoxy, haloalkoxy, alkylthio, alkylsulfinyl, alkylsulfonyl, haloalkylsulfonyl, alkenyl and alkynyl groups and corresponding group moieties in larger groups such as alkoxyalkyl, (di)alkylaminocarbonyl, alkoxycarbonyl, cycloalkoxyalkyl, alkoxycarbonylalkyl etc. can be straight-chain or branched, the prefix Cₙ-Cₘ in each case indicating the possible number of carbon atoms in the group. Halogenated substituents preferably carry one, two, three, four or five identical or different halogen atoms. The meaning halogen is in each case fluorine, chlorine, bromine or iodine, preferably fluorine or chlorine.

In addition, the following are, for example:

C₁-C₄-alkyl: CH₃, C₂H₅, n-propyl, CH(CH₃)₂, n-butyl, CH(CH₃)—C₂H₅, CH₂—CH(CH₃)₂ and C(CH₃)₃;

C₁-C₄-haloalkyl: a C₁-C₄-alkyl radical as mentioned above, which is partially or completely substituted by fluorine, chlorine, bromine and/or iodine, i.e., for example, CH₂F, CHF₂, CF₃, CH₂Cl, dichloromethyl, trichloromethyl, chlorofluoromethyl, dichlorofluoromethyl, chlorodifluoromethyl, 2-fluoroethyl, 2-chloroethyl, 2-bromoethyl, 2-iodoethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, C₂F₅, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, 2,2,3,3,3-pentafluoropropyl, heptafluoropropyl, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl or nonafluorobutyl;

hydroxy-C₁-C₄-alkyl: e.g. hydroxymethyl, 2-hydroxyeth-1-yl, 2-hydroxyprop-1-yl, 3-hydroxyprop-1-yl, 1-hydroxyprop-2-yl, 2-hydroxybut-1-yl, 3-hydroxybut-1-yl, 4-hydroxybut-1-yl, 1-hydroxybut-2-yl, 1-hydroxybut-3-yl, 2-hydroxybut-3-yl, 1-hydroxy-2-methylprop-3-yl, 2-hydroxy-2-methylprop-3-yl or 2-hydroxymethylprop-2-yl, in particular 2-hydroxyethyl;

cyano-C₁-C₄-alkyl: e.g. cyanomethyl, 1-cyanoeth-1-yl, 2-cyanoeth-1-yl, 1-cyanoprop-1-yl, 2-cyanoprop-1-yl, 3-cyanoprop-1-yl, 1-cyanoprop-2-yl, 2-cyanoprop-2-yl, 1-cyanobut-1-yl, 2-cyanobut-1-yl, 3-cyanobut-1-yl, 4-cyanobut-1-yl, 1-cyanobut-2-yl, 2-cyanobut-2-yl, 1-cyanobut-3-yl, 2-cyanobut-3-yl, 1-cyano-2-methylprop-3-yl, 2-cyano-2-methylprop-3-yl, 3-cyano-2-methylprop-3-yl or 2-cyanomethylprop-2-yl, in particular cyanomethyl or 2-cyanoethyl;

amino-C₁-C₄-alkyl: CH₂NH₂, 1-aminoethyl, 2-aminoethyl, 1-aminoprop-1-yl, 2-aminoprop-1-yl, 3-aminoprop-1-yl, 1-aminobut-1-yl, 2-aminobut-1-yl, 3-aminobut-1-yl, 4-aminobut-1-yl, 1-aminobut-2-yl, 2-aminobut-2-yl, 3-aminobut-2-yl, 4-aminobut-2-yl, 1-(CH₂NH₂)eth-1-yl, 1-(CH₂NH₂)-1-(CH₃)-eth-1-yl or 1-(CH₂NH₂)prop-1-yl;

C₁-C₄-alkylamino-C₁-C₄-alkyl: C₁-C₄-alkyl substituted by C₁-C₄-alkylamino such as H₃C—NH—, H₅C₂—NH—, n-propyl-NH—, 1-methylethyl-NH, n-butyl-NH—, 1-methylpropyl-NH—, 2-methylpropyl-NH— and 1,1-dimethylethyl-NH—, i.e., for example, CH₂CH₂—NH—CH₃, CH₂CH₂—NH—C₂H₅, CH₂CH₂—NH—CH₂CH₂CH₃, CH₂CH₂—NHCH(CH₃)₂ etc.

di(C₁-C₄-alkyl)amino-C₁-C₄-alkyl: C₁-C₄-alkyl substituted by di(C₁-C₄-alkyl)amino, i.e., for example, CH₂N(CH₃)₂, CH₂N(C₂H₅)₂, N,N-dipropylaminomethyl, N,N-di[CH(CH₃)₂]aminomethyl, N,N-dibutylaminomethyl, N,N-di(1-methylpropyl)aminomethyl, N,N-di(2-methylpropyl)aminomethyl, N,N-di[C(CH₃)₃]aminomethyl, N-ethyl-N-methylaminomethyl, N-methyl-N-propylaminomethyl, N-methyl-N—[CH(CH₃)₂]aminomethyl, N-butyl-N-methylaminomethyl, N-methyl-N-(1-methylpropyl)aminomethyl, N-methyl-N-(2-methylpropyl)aminomethyl, N—[C(CH₃)₃]—N-methylaminomethyl, N-ethyl-N-propylaminomethyl, N-ethyl-N—[CH(CH₃)₂]aminomethyl, N-butyl-N-ethylaminomethyl, N-ethyl-N-(1-methylpropyl)aminomethyl, N-ethyl-N-(2-methylpropyl)aminomethyl, N-ethyl-N—[C(CH₃)₃]aminomethyl, N—[CH(CH₃)₂]—N-propylaminomethyl, N-butyl-N-propylaminomethyl, N-(1-methylpropyl)-N-propylaminomethyl, N-(2-methylpropyl)-N-propylaminomethyl, N—[C(CH₃)₃]—N-propylaminomethyl, N-butyl-N-(1-methylethyl)aminomethyl, N—[CH(CH₃)₂]—N-(1-methylpropyl)aminomethyl, N—[CH(CH₃)₂]—N-(2-methylpropyl)aminomethyl, N—[C(CH₃)₃]—N—[CH(CH₃)₂]aminomethyl, N-butyl-N-(1-methylpropyl)aminomethyl, N-butyl-N-(2-methylpropyl)aminomethyl, N-butyl-N—[C(CH₃)₃]aminomethyl, N-(1-methylpropyl)-N-(2-methylpropyl)aminomethyl, N—[C(CH₃)₃]—N-(1-methylpropyl)aminomethyl, N—[C(CH₃)₃]—N-(2-methylpropyl)aminomethyl, N,N-dimethylaminoethyl, N,N-diethylaminoethyl, N,N-di(n-propyl)aminoethyl, N,N-di[CH(CH₃)₂]aminoethyl, N,N-dibutylaminoethyl, N,N-di(1-methylpropyl)aminoethyl, N,N-di(2-methylpropyl)aminoethyl, N,N-di[C(CH₃)₃]aminoethyl, N-ethyl-N-methylaminoethyl, N-methyl-N-propylaminoethyl, N-methyl-N—[CH(CH₃)₂]aminoethyl, N-butyl-N-methylaminoethyl, N-methyl-N-(1-methylpropyl)aminoethyl, N-methyl-N-(2-methylpropyl)aminoethyl, N—[C(CH₃)₃]—N-methylaminoethyl, N-ethyl-N-propylaminoethyl, N-ethyl-N—[CH(CH₃)₂]aminoethyl, N-butyl-N-ethylaminoethyl, N-ethyl-N-(1-methylpropyl)aminoethyl, N-ethyl-N-(2-methylpropyl)aminoethyl, N-ethyl-N—[C(CH₃)₃]aminoethyl, N—[CH(CH₃)₂]—N-propylaminoethyl, N-butyl-N-propylaminoethyl, N-(1-methylpropyl)-N-propylaminoethyl, N-(2-methylpropyl)-N-propylaminoethyl, N—[C(CH₃)₃]—N-propylaminoethyl, N-butyl-N—[CH(CH₃)₂]aminoethyl, N—[CH(CH₃)₂]—N-(1-methylpropyl)aminoethyl, N—[CH(CH₃)₂]—N-(2-methylpropyl)aminoethyl, N—[C(CH₃)₃]—N—[CH(CH₃)₂]aminoethyl, N-butyl-N-(1-methylpropyl)aminoethyl, N-butyl-N-(2-methylpropyl)aminoethyl, N-butyl-N—[C(CH₃)₃]aminoethyl, N-(1-methylpropyl)-N-(2-methylpropyl)aminoethyl, N—[C(CH₃)₃]—N-(1-methylpropyl)aminoethyl or N—[C(CH₃)₃]—N-(2-methylpropyl)aminoethyl, in particular N,N-dimethylaminoethyl or N,N-diethylaminoethyl;

C₁-C₄-alkoxy-C₁-C₄-alkyl: CH₂—OCH₃, CH₂—OC₂H₅, n-propoxymethyl, CH₂—OCH(CH₃)₂, n-butoxymethyl, (1-methylpropoxy)methyl, (2-methylpropoxy)methyl, CH₂—OC(CH₃)₃, 2-(methoxy)ethyl, 2-(ethoxy)ethyl, 2-(n-propoxy)ethyl, 2-(1-methylethoxy)ethyl, 2-(n-butoxy)ethyl, 2-(1-methylpropoxy)ethyl, 2-(2-methylpropoxy)ethyl, 2-(1,1-dimethylethoxy)ethyl, 2-(methoxy)propyl, 2-(ethoxy)propyl, 2-(n-propoxy)propyl, 2-(1-methylethoxy)propyl, 2-(n-butoxy)propyl, 2-(1-methylpropoxy)propyl, 2-(2-methylpropoxy)propyl, 2-(1,1-dimethylethoxy)propyl, 3-(methoxy)propyl, 3-(ethoxy)propyl, 3-(n-propoxy)propyl, 3-(1-methylethoxy)propyl, 3-(n-butoxy)propyl, 3-(1-methylpropoxy)propyl, 3-(2-methylpropoxy)propyl, 3-(1,1-dimethylethoxy)propyl, 2-(methoxy)butyl, 2-(ethoxy)butyl, 2-(n-propoxy)butyl, 2-(1-methylethoxy)butyl, 2-(n-butoxy)butyl, 2-(1-methylpropoxy)butyl, 2-(2-methylpropoxy)butyl, 2-(1,1-dimethylethoxy)butyl, 3-(methoxy)butyl, 3-(ethoxy)butyl, 3-(n-propoxy)butyl, 3-(1-methylethoxy)butyl, 3-(n-butoxy)butyl, 3-(1-methylpropoxy)butyl, 3-(2-methylpropoxy)butyl, 3-(1,1-dimethylethoxy)butyl, 4-(methoxy)butyl, 4-(ethoxy)butyl, 4-(n-propoxy)butyl, 4-(1-methylethoxy)butyl, 4-(n-butoxy)butyl, 4-(1-methylpropoxy)butyl, 4-(2-methylpropoxy)butyl or 4-(1,1-dimethylethoxy)butyl, preferably CH₂—OCH₃, CH₂—OC₂H₅, 2-(OCH₃)ethyl or 2-(OC₂H₅)ethyl;

C₁-C₄-haloalkoxy-C₁-C₄-alkyl: C₁-C₄-alkyl substituted by C₁-C₄-haloalkoxy such as OCH₂F, OCHF₂, OCF₃, OCH₂Cl, OCH(Cl)₂, OC(Cl)₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, OC₂F₅, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy, preferably OCHF₂, OCF₃, dichlorofluoromethoxy, chlorodifluoromethoxy or 2,2,2-trifluoroethoxy, i.e., for example, 2-(OCHF₂)ethyl, 2-(OCF₃)ethyl or 2-(OC₂F₅)ethyl;

hydroxycarbonyl-C₁-C₄-alkoxy-C₁-C₄-alkyl: C₁-C₄-alkoxy-C₁-C₄-alkyl substituted by COOH in the alkoxy moiety, i.e., —CH₂—O—CH₂COOH, —CH₂CH₂—O—CH₂COOH, —CH₂CH₂CH₂—O—CH₂COOH, —CH₂CH(CH₃)—O—CH₂COOH, —CH(CH₃)CH₂—O—CH₂COOH, —CH₂—O—CH₂CH₂COOH, —CH₂CH₂—O—CH₂CH₂COOH, —CH₂CH₂CH₂—O—CH₂CH₂COOH, —CH₂CH(CH₃)—O—CH₂CH₂COOH, —CH(CH₃)CH₂—O—CH₂CH₂COOH, —CH₂—O—CH(CH₃)COOH, —CH₂CH₂—O—CH(CH₃)COOH, —CH₂CH₂CH₂—O—CH(CH₃)COOH, —CH₂CH(CH₃)—O—CH(CH₃)COOH, —CH(CH₃)CH₂—O—CH(CH₃)COOH;

aminocarbonyl-C₁-C₄-alkoxy-C₁-C₄-alkyl: C₁-C₄-alkoxy-C₁-C₄-alkyl substituted by CONH₂ in the alkoxy moiety, i.e., —CH₂—O—CH₂CONH₂, —CH₂CH₂—O—CH₂CONH₂, —CH₂CH₂CH₂—O—CH₂CONH₂, —CH₂CH(CH₃)-O—CH₂CONH₂, —CH(CH₃)CH₂—O—CH₂CONH₂, —CH₂—O—CH₂CH₂CONH₂, —CH₂CH₂—O—CH₂CH₂CONH₂, —CH₂CH₂CH₂—O—CH₂CH₂CONH₂, —CH₂CH(CH₃)—O—CH₂CH₂CONH₂, —CH(CH₃)CH₂—O—CH₂CH₂CONH₂, —CH₂—O—CH(CH₃)CONH₂, —CH₂CH₂—O—CH(CH₃)CONH₂, —CH₂CH₂CH₂—O—CH(CH₃)CONH₂, —CH₂CH(CH₃)—O—CH(CH₃)CONH₂, —CH(CH₃)CH₂—O—CH(CH₃)CONH₂;

C₁-C₄-alkylthio-C₁-C₄-alkyl: CH₂—SCH₃, CH₂—SC₂H₅, n-propylthiomethyl, CH₂—SCH(CH₃)₂, n-butylthiomethyl, (1-methylpropylthio)methyl, (2-methylpropylthio)methyl, CH₂—SC(CH₃)₃, 2-(methylthio)ethyl, 2-(ethylthio)ethyl, 2-(n-propylthio)ethyl, 2-(1-methylethylthio)ethyl, 2-(n-butylthio)ethyl, 2-(1-methylpropylthio)ethyl, 2-(2-methylpropylthio)ethyl, 2-(1,1-dimethylethylthio)ethyl, 2-(methylthio)propyl, 2-(ethylthio)propyl, 2-(n-propylthio)propyl, 2-(1-methylethylthio)propyl, 2-(n-butylthio)propyl, 2-(1-methylpropylthio)propyl, 2-(2-methylpropylthio)propyl, 2-(1,1-dimethylethylthio)propyl, 3-(methylthio)propyl, 3-(ethylthio)propyl, 3-(n-propylthio)propyl, 3-(1-methylethylthio)propyl, 3-(n-butylthio)propyl, 3-(1-methylpropylthio)propyl, 3-(2-methylpropylthio)propyl, 3-(1,1-dimethylethylthio)propyl, 2-(methylthio)butyl, 2-(ethylthio)butyl, 2-(n-propylthio)butyl, 2-(1-methylethylthio)butyl, 2-(n-butylthio)butyl, 2-(1-methylpropylthio)butyl, 2-(2-methylpropylthio)butyl, 2-(1,1-dimethylethylthio)butyl, 3-(methylthio)butyl, 3-(ethylthio)butyl, 3-(n-propylthio)butyl, 3-(1-methylethylthio)butyl, 3-(n-butylthio)butyl, 3-(1-methylpropylthio)butyl, 3-(2-methylpropylthio)butyl, 3-(1,1-dimethylethylthio)butyl, 4-(methylthio)butyl, 4-(ethylthio)butyl, 4-(n-propylthio)butyl, 4-(1-methylethylthio)butyl, 4-(n-butylthio)butyl, 4-(1-methylpropylthio)butyl, 4-(2-methylpropylthio)butyl or 4-(1,1-dimethylethylthio)butyl, preferably CH₂—SCH₃, CH₂—SC₂H₅, 2-(SCH₃)ethyl or 2-(SC₂H₅)ethyl;

C₁-C₄-alkylsulfinyl-C₁-C₄-alkyl: a C₁-C₄-alkyl substituted by a C₁-C₄-alkylsulfinyl radical such as SO—CH₃, SO—C₂H₅, SO—CH₂—C₂H₅, SO—CH(CH₃)₂, SO-(n-C₄H₉), SO—CH(CH₃)—C₂H₅, SO—CH₂—CH(CH₃)₂ or SO—C(CH₃)₃, i.e., for example, CH₂SO—CH₃, CH₂SO—C₂H₅, CH₂SO—CH₂—C₂H₅, CH₂SO—CH(CH₃), CH₂SO—CH₂CH₂CH₂CH₃, CH₂SO—CH(CH₃)—C₂H₅, CH₂SO—CH₂—CH(CH₃)₂, CH₂CH₂SO—CH₃, CH₂CH₂SO—C₂H₅, CH₂CH₂SO—CH₂—C₂H₅, CH₂CH₂SO—CH(CH₃)₂, CH₂CH₂SO—CH₂CH₂CH₂CH₃, CH₂CH₂SO—CH(CH₃)—C₂H₅, CH₂CH₂SO—CH₂—CH(CH₃)₂;

C₁-C₄-alkylsulfonyl-C₁-C₄-alkyl: a C₁-C₄-alkyl substituted by a C₁-C₄-alkylsulfonyl radical such as SO₂—CH₃, SO₂—C₂H₅, SO₂—CH₂—C₂H₅, SO₂—CH(CH₃)₂, n-butylsulfonyl, SO₂—CH(CH₃)—C₂H₅, SO₂—CH₂—CH(CH₃)₂ or SO₂—C(CH₃)₃, preferably SO₂—CH₃ or SO₂—C₂H₅, i.e., for example, CH₂SO₂—CH₃, CH₂SO₂—C₂H₅, CH₂SO₂—CH₂—C₂H₅, CH₂SO₂—CH(CH₃)₂, CH₂SO₂—CH₂CH₂CH₂CH₃, CH₂SO₂—CH(CH₃)—C₂H₅, CH₂SO₂—CH₂—CH(CH₃)₂, CH₂CH₂SO₂—CH₃, CH₂CH₂SO₂—C₂H₅, CH₂CH₂SO₂—CH₂—C₂H₅, CH₂CH₂SO₂—CH(CH₃)₂, CH₂CH₂SO₂—CH₂CH₂CH₂CH₃, CH₂CH₂SO₂—CH(CH₃)—C₂H₅, CH₂CH₂SO₂—CH₂—CH(CH₃)₂;

hydroxycarbonyl-C₁-C₄-alkyl: CH₂COOH, 1-(COOH)ethyl, 2-(COOH)ethyl, 1-(COOH)prop-1-yl, 2-(COOH)prop-1-yl, 3-(COOH)prop-1-yl, 1-(COOH)but-1-yl, 2-(COOH)but-1-yl, 3-(COOH)but-1-yl, 4-(COOH)but-1-yl, 1-(COOH)but-2-yl, 2-(COOH)but-2-yl, 3-(COOH)but-2-yl, 4-(COOH)but-2-yl, 1-(CH₂COOH)eth-1-yl, 1-(CH₂COOH)-1-(CH₃)eth-1-yl or 1-(CH₂COOH)prop-1-yl;

(C₁-C₄-alkoxy)carbonyl-C₁-C₄-alkyl: C₁-C₄-alkyl substituted by (C₁-C₄-alkoxy)carbonyl such as CO—OCH₃, CO—OC₂H₅, CO—OCH₂—C₂H₅, CO—OCH(CH₃)₂, n-butoxycarbonyl, CO—OCH(CH₃)—C₂H₅, CO—OCH₂—CH(CH₃)₂ or CO—OC(CH₃)₃, preferably CO—OCH₃ or CO—OC₂H₅, i.e., for example, CH₂—CO—OCH₃, CH₂—CO—OC₂H₅, CH₂—CO—OCH₂—C₂H₅, CH₂—CO—OCH(CH₃)₂, n-butoxycarbonylmethyl, CH₂—CO—OCH(CH₃)—C₂H₅, CH₂—CO—OCH₂—CH(CH₃)₂, CH₂—CO—OC(CH₃)₃, 1-(CO—OCH₃)ethyl, 1-(CO—OC₂H₅)ethyl, 1-(CO—OCH₂—C₂H₅)ethyl, 1-[CH(CH₃)₂]ethyl, 1-(n-butoxycarbonyl)ethyl, 1-[1-methylpropoxycarbonyl]ethyl, 1-[2-methylpropoxycarbonyl]ethyl, 2-(CO—OCH₃)ethyl, 2-(CO—OC₂H₅)ethyl, 2-(CO—OCH₂—C₂H₅)ethyl, 2-[CO—OCH(CH₃)₂]ethyl, 2-(n-butoxycarbonyl)ethyl, 2-[1-methylpropoxycarbonyl]ethyl, 2-[2-methylpropoxycarbonyl]ethyl, 2-[CO—OC(CH₃)₃]ethyl, 2-(CO—OCH₃)propyl, 2-(CO—OC₂H₅)propyl, 2-(CO—OCH₂—C₂H₅)propyl, 2-[CO—OCH(CH₃)₂]propyl, 2-(n-butoxycarbonyl)propyl, 2-[1-methylpropoxycarbonyl]propyl, 2-[2-methylpropoxycarbonyl]propyl, 2-[CO—OC(CH₃)₃]propyl, 3-(CO—OCH₃)propyl, 3-(CO—OC₂H₅)propyl, 3-(CO—OCH₂—C₂H₅)propyl, 3-[CO—OCH(CH₃)₂]propyl, 3-(n-butoxycarbonyl)propyl, 3-[1-methylpropoxycarbonyl]propyl, 3-[2-methylpropoxycarbonyl]propyl, 3-[CO—OC(CH₃)₃]propyl, 2-(CO—OCH₃)butyl, 2-(CO—OC₂H₅)butyl, 2-(CO—OCH₂—C₂H₅)butyl, 2-[CO—OCH(CH₃)₂]butyl, 2-(n-butoxycarbonyl)butyl, 2-[1-methylpropoxycarbonyl]butyl, 2-[2-methylpropoxycarbonyl]butyl, 2-[CO—OC(CH₃)₃]butyl, 3-(CO—OCH₃)butyl, 3-(CO—OC₂H₅)butyl, 3-(CO—OCH₂—C₂H₅)butyl, 3-[CO—OCH(CH₃)₂]butyl, 3-(n-butoxycarbonyl)butyl, 3-[1-methylpropoxycarbonyl]butyl, 3-[2-methylpropoxycarbonyl]butyl, 3-[CO—OC(CH₃)₃]butyl, 4-(CO—OCH₃)butyl, 4-(CO—OC₂H₅)butyl, 4-(CO—OCH₂—C₂H₅)butyl, 4-[CO—OCH(CH₃)₂]butyl, 4-(n-butoxycarbonyl)butyl, 4-[1-methylpropoxycarbonyl]butyl, 4-[2-methylpropoxycarbonyl]butyl or 4-[CO—OC(CH₃)₃]butyl, preferably CH₂—CO—OCH₃, CH₂—CO—OC₂H₅, 1-(CO—OCH₃)ethyl or 1-(CO—OC₂H₅)ethyl;

aminocarbonyl-C₁-C₄-alkyl: CH₂CONH₂, 1-(CONH₂)ethyl, 2-(CONH₂)ethyl, 1-(CONH₂)prop-1-yl, 2-(CONH₂)prop-1-yl, 3-(CONH₂)prop-1-yl, 1-(CONH₂)but-1-yl, 2-(CONH₂)but-1-yl, 3-(CONH₂)but-1-yl, 4-(CONH₂)but-1-yl, 1-(CONH₂)but-2-yl, 2-(CONH₂)but-2-yl, 3-(CONH₂)but-2-yl, 4-(CONH₂)but-2-yl, 1-(CH₂CONH₂)eth-1-yl, 1-(CH₂CONH₂)-1-(CH₃)eth-1-yl or 1-(CH₂CONH₂)prop-1-yl;

(C₁-C₄-alkylamino)carbonyl-C₁-C₄-alkyl: C₁-C₄-alkyl substituted by (C₁-C₄-alkylamino)carbonyl such as CO—NH—CH₃, CO—NH—C₂H₅, n-propylaminocarbonyl, CO—NH—CH(CH₃)₂, CO—NH—CH₂CH₂—C₂H₅, CO—NH—CH(CH₃)—C₂H₅, CO—NH—CH₂—CH(CH₃)₂ or CO—NH—C(CH₃)₃, preferably CO—NH—CH₃ or CO—NH—C₂H₅, as, for example, CH₂—CO—NH—CH₃, CH₂—CO—NH—C₂H₅, CH₂—CO—NH—CH₂—C₂H₅, CH₂—CO—NH—CH(CH₃)₂, CH₂—CO—NH—CH₂CH₂—C₂H₅, CH₂—CO—NH—CH(CH₃)—C₂H₅, CH₂—CO—NH—CH₂—CH(CH₃)₂, CH₂—CO—NH—C(CH₃)₃, CH(CH₃)—CO—NH—CH₃, CH(CH₃)—CO—NH—C₂H₅, 2-(CO—NH—CH₃)ethyl, 2-(CO—NH—C₂H₅)ethyl, 2-(CO—NH—CH₂—C₂H₅)ethyl, 2-[CH₂—CO—NH—CH(CH₃)₂]ethyl, 2-(CO—NH—CH₂CH₂—C₂H₅)ethyl, 2-[CO—NH—CH(CH₃)—C₂H₅]ethyl, 2-[CO—NH—CH₂—CH(CH₃)₂]ethyl, 2-[CO—NH—C(CH₃)₃]ethyl, 2-(CO—NH—CH₃)propyl, 2-(CO—NH—C₂H₅)propyl, 2-(CO—NH—CH₂—C₂H₅)propyl, 2-[CH₂—CO—NH—CH(CH₃)₂]propyl, 2-(CO—NH—CH₂CH₂—C₂H₅)propyl, 2-[CO—NH—CH(CH₃)—C₂H₅]propyl, 2-[CO—NH—CH₂—CH(CH₃)₂]propyl, 2-[CO—NH—C(CH₃)₃]propyl, 3-(CO—NH—CH₃)propyl, 3-(CO—NH—C₂H₅)propyl, 3-(CO—NH—CH₂—C₂H₅)propyl, 3-[CH₂—CO—NH—CH(CH₃)₂]propyl, 3-(CO—NH—CH₂CH₂—C₂H₅)propyl, 3-[CO—NH—CH(CH₃)—C₂H₅]propyl, 3-[CO—NH—CH₂—CH(CH₃)₂]propyl, 3-[CO—NH—C(CH₃)₃]propyl, 2-(CO—NH—CH₃)butyl, 2-(CO—NH—C₂H₅)butyl, 2-(CO—NH—CH₂—C₂H₅)butyl, 2-[CH₂—CO—NH—CH(CH₃)₂]butyl, 2-(CO—NH—CH₂CH₂—C₂H₅)butyl, 2-[CO—NH—CH(CH₃)—C₂H₅]butyl, 2-[CO—NH—CH₂—CH(CH₃)₂]butyl, 2-[CO—NH—C(CH₃)₃]butyl, 3-(CO—NH—CH₃)butyl, 3-(CO—NH—C₂H₅)butyl, 3-(CO—NH—CH₂—C₂H₅)butyl, 3-[CH₂—CO—NH—CH(CH₃)₂]butyl, 3-(CO—NH—CH₂CH₂—C₂H₅)butyl, 3-[CO—NH—CH(CH₃)—C₂H₅]butyl, 3-[CO—NH—CH₂—CH(CH₃)₂]butyl, 3-[CO—NH—C(CH₃)₃]butyl, 4-(CO—NH—CH₃)butyl, 4-(CO—NH—C₂H₅)butyl, 4-(CO—NH—CH₂—C₂H₅)butyl, 4-[CH₂—CO—NH—CH(CH₃)₂]butyl, 4-(CO—NH—CH₂CH₂—C₂H₅)butyl, 4-[CO—NH—CH(CH₃)—C₂H₅]butyl, 4-[CO—NH—CH₂—CH(CH₃)₂]butyl or 4-[CO—NH—C(CH₃)₃]butyl, preferably CH₂—CO—NH—CH₃, CH₂—CO—NH—C₂H₅, CH(CH₃)—CO—NH—CH₃ or CH(CH₃)—CO—NH—C₂H₅;

di(C₁-C₄-alkyl)aminocarbonyl-C₁-C₄-alkyl: C₁-C₄-alkyl substituted by di(C₁-C₄-alkyl)aminocarbonyl such as CO—N(CH₃)₂, CO—N(C₂H₅), CO—N(CH₂—C₂H₅)₂, CO—N[CH(CH₃)₂]₂, N,N-dibutylaminocarbonyl, CO—N[CH(CH₃)—C₂H₅]₂, CO—N[CH₂—CH(CH₃)₂]₂, CO—N[C(CH₃)₃]₂, N-ethyl-N-methylaminocarbonyl, N-methyl-N-propylaminocarbonyl, N-methyl-N—[CH(CH₃)₂]aminocarbonyl, N-butyl-N-methylaminocarbonyl, N-methyl-N-(1-methylpropyl)aminocarbonyl, N-methyl-N-(2-methylpropyl)aminocarbonyl, N—[C(CH₃)₃]—N-methylaminocarbonyl, N-ethyl-N-propylaminocarbonyl, N-ethyl-N—[CH(CH₃)₂]aminocarbonyl, N-butyl-N-ethylaminocarbonyl, N-ethyl-N-(1-methylpropyl)aminocarbonyl, N-ethyl-N-(2-methylpropyl)aminocarbonyl, N-ethyl-N—[C(CH₃)₃]aminocarbonyl, N—[CH(CH₃)₂]—N-propylaminocarbonyl, N-butyl-N-propylaminocarbonyl, N-(1-methylpropyl)-N-propylaminocarbonyl, N-(2-methylpropyl)-N-propylaminocarbonyl, N—[C(CH₃)₃]—N-propylaminocarbonyl, N-butyl-N—[CH(CH₃)₂]aminocarbonyl, N—[CH(CH₃)₂]—N-(1-methylpropyl)aminocarbonyl, N—[CH(CH₃)₂]—N-(2-methylpropyl)aminocarbonyl, N—[C(CH₃)₃]—N—[CH(CH₃)₂]aminocarbonyl, N-butyl-N-(1-methylpropyl)aminocarbonyl, N-butyl-N-(2-methylpropyl)aminocarbonyl, N-butyl-N—[C(CH₃)₃]aminocarbonyl, N-(1-methylpropyl)-N-(2-methylpropyl)aminocarbonyl, N—[C(CH₃)₃]—N-(1-methylpropyl)aminocarbonyl or N—[C(CH₃)₃]—N-(2-methylpropyl)aminocarbonyl, preferably CO—N(CH₃)₂ or CO—N(C₂H₅)₂, as, for example, N,N-dipropylaminocarbonylmethyl, N,N-di[CH(CH₃)₂]aminocarbonylmethyl, N,N-dibutylaminocarbonylmethyl, N,N-di(1-methylpropyl)aminocarbonylmethyl, N,N-di(2-methylpropyl)aminocarbonylmethyl, N-ethyl-N-methylaminocarbonylmethyl, N-methyl-N-propylaminocarbonylmethyl, N-methyl-N—[CH(CH₃)₂]aminocarbonylmethyl, N-butyl-N-methylaminocarbonylmethyl, N-methyl-N-(1-methylpropyl)aminocarbonylmethyl, N-methyl-N-(2-methylpropyl)aminocarbonylmethyl, N-ethyl-N-propylaminocarbonylmethyl, N-ethyl-N—[CH(CH₃)₂]aminocarbonylmethyl, N-butyl-N-ethylaminocarbonylmethyl, N-ethyl-N-(1-methylpropyl)aminocarbonylmethyl, N-ethyl-N-(2-methylpropyl)aminocarbonylmethyl, N-ethyl-N—[C(CH₃)₃]aminocarbonylmethyl, N—[CH(CH₃)₂]—N-propylaminocarbonylmethyl, N-butyl-N-propylaminocarbonylmethyl, N-(1-methylpropyl)-N-propylaminocarbonylmethyl, N-(2-methylpropyl)-N-propylaminocarbonylmethyl, N-butyl-N-(1-methylethyl)aminocarbonylmethyl, N—[CH(CH₃)₂]—N-(1-methylpropyl)aminocarbonylmethyl, N—[CH(CH₃)₂]—N-(2-methylpropyl)aminocarbonylmethyl, N-butyl-N-(1-methylpropyl)aminocarbonylmethyl, N-butyl-N-(2-methylpropyl)aminocarbonylmethyl, N-(1-methylpropyl)-N-(2-methylpropyl)aminocarbonylmethyl, N,N-dimethylaminocarbonylethyl, N,N-diethylaminocarbonylethyl, N,N-di(n-propyl)aminocarbonylethyl, N,N-di[CH(CH₃)₂]aminocarbonylethyl, N,N-dibutylaminocarbonylethyl, N,N-di(1-methylpropyl)aminocarbonylethyl, N,N-di(2-methylpropyl)aminocarbonylethyl, N-ethyl-N-methylaminocarbonylethyl, N-methyl-N-propylaminocarbonylethyl, N-methyl-N—[CH(CH₃)₂]aminocarbonylethyl, N-butyl-N-methylaminocarbonylethyl, N-methyl-N-(1-methylpropyl)aminocarbonylethyl, N-methyl-N-(2-methylpropyl)aminocarbonylethyl, N—[C(CH₃)₃]—N-methylaminocarbonylethyl, N-ethyl-N-propylaminocarbonylethyl, N-ethyl-N—[CH(CH₃)₂]aminocarbonylethyl, N-butyl-N-ethylaminocarbonylethyl, N-ethyl-N-(1-methylpropyl)aminocarbonylethyl, N-ethyl-N-(2-methylpropyl)aminocarbonylethyl, N—[CH(CH₃)₂]—N-propylaminocarbonylethyl, N-butyl-N-propylaminocarbonylethyl, N-(1-methylpropyl)-N-propylaminocarbonylethyl, N-(2-methylpropyl)-N-propylaminocarbonylethyl, N-butyl-N—[CH(CH₃)₂]aminocarbonylethyl, N—[CH(CH₃)₂]—N-(1-methylpropyl)aminocarbonylethyl, N—[CH(CH₃)₂]—N-(2-methylpropyl)aminocarbonylethyl, N-butyl-N-(1-methylpropyl)aminocarbonylethyl, N-butyl-N-(2-methylpropyl)aminocarbonylethyl, N-(1-methylpropyl)-N-(2-methylpropyl)aminocarbonylethyl, in particular CH₂CO—N(CH₃)₂, CH₂CO—N (C₂H₅)₂, CH₂CH₂CO—N(CH₃)₂, CH₂CH₂CO—N(C₂H₅)₂, CHCH₃CO—N(CH₃)₂, CHCH₃CO—N(C₂H₅)₂, C(CH₃)₂CO—N(CH₃)₂ or C(CH₃)₂CO—N(C₂H₅)₂;

C₁-C₄-alkoxy-C₁-C₄-alkoxy-C₁-C₄-alkyl: C₁-C₄-alkoxy-C₁-C₄-alkyl substituted by C₁-C₄-alkoxy in the alkoxy moiety, e.g. —CH₂CH₂—O—CH₂CH₂—O—CH₃;

(C₁-C₄-alkoxy)carbonyl-C₁-C₄-alkoxy-C₁-C₄-alkyl: C₁-C₄-alkoxy-C₁-C₄-alkyl substituted by C₁-C₄-alkoxycarbonyl in the alkoxy moiety, e.g. —CH₂CH₂—O—CH₂C(O)—OCH₃, —CH₂CH₂—O—CH(CH₃)C(O)—OCH₃, —CH₂CH₂—O—CH₂C(O)—OCH₂CH₃, —CH₂CH₂—O—CH(CH₃)C(O)—OCH₂CH₃;

(C₁-C₄-alkylthio)carbonyl-C₁-C₄-alkoxy-C₁-C₄-alkyl: C₁-C₄-alkoxy-C₁-C₄-alkyl substituted by C₁-C₄-alkylthiocarbonyl in the alkoxy moiety, e.g. —CH₂CH₂—O—CH₂C(O)—SCH₃; or —CH₂CH₂—O—CH(CH₃)C(O)—SCH₃;

(C₁-C₄-alkyl)aminocarbonyl-C₁-C₄-alkoxy-C₁-C₄-alkyl: C₁-C₄-alkoxy-C₁-C₄-alkyl substituted by (C₁-C₄-alkyl)aminocarbonyl in the alkoxy moiety, e.g. —CH₂CH₂—O—CH₂C(O)—NHCH₃ or —CH₂CH₂—O—CH(CH₃)—C(O)—NHCH₃;

di(C₁-C₄-alkyl)aminocarbonyl-C₁-C₄-alkoxy-C₁-C₄-alkyl: C₁-C₄-alkoxy-C₁-C₄-alkyl substituted by di(C₁-C₄-alkyl)aminocarbonyl in the alkoxy moiety, e.g. —CH₂CH₂—O—CH₂—C(O)—N(CH₃)₂ or —CH₂CH₂—O—CH(CH₃)—C(O)—N(CH₃)₂;

C₃-C₄-alkenyl and the C₃-C₄-alkenyl moieties in C₃-C₄-alkenyloxy-C₁-C₄-alkyl and C₃-C₄-alkenyloxycarbonyl-C₁-C₄-alkyl: unsaturated, straight-chain or branched hydrocarbon radicals having 3 to 4 carbon atoms and a double bond in any desired position, e.g. 1-propenyl, 2-propenyl, 1-methylethenyl, 1-buten-1-yl, 1-buten-2-yl, 1-buten-3-yl, 2-buten-1-yl, 1-methylprop-1-en-1-yl, 2-methylprop-1-en-1-yl, 1-methylprop-2-en-1-yl, 2-methylprop-2-en-1-yl;

C₃-C₄-alkynyl and the C₃-C₄-alkynyl moieties in C₃-C₄-alkynyloxy-C₁-C₄-alkyl and C₃-C₄-alkynyloxycarbonyl-C₁-C₄-alkyl: straight-chain or branched hydrocarbon groups having 3 to 4 carbon atoms and a triple bond in any desired position, e.g. 1-propynyl, 2-propynyl (═propargyl), 1-butynyl, 2-butynyl, 3-butynyl and 1-methyl-2-propynyl;

C₃-C₄-alkenyloxy-C₁-C₄-alkyl: C₁-C₄-alkyl substituted by C₃-C₄-alkenyloxy such as allyloxy, but-1-en-3-yloxy, but-1-en-4-yloxy, but-2-en-1-yloxy, 1-methylprop-2-enyloxy or 2-methylprop-2-enyloxy, i.e., for example, allyloxymethyl, 2-allyloxyethyl or but-1-en-4-yloxymethyl, in particular 2-allyloxyethyl;

C₃-C₄-alkenyloxycarbonyl-C₁-C₄-alkyl: C₁-C₄-alkyl substituted by C₃-C₄-alkenyloxycarbonyl such as allyloxycarbonyl, but-1-en-3-yloxycarbonyl, but-1-en-4-yloxycarbonyl, but-2-en-1-yloxycarbonyl, 1-methylprop-2-enyloxycarbonyl or 2-methylprop-2-enyloxycarbonyl, i.e., for example, allyloxycarbonylmethyl, 2-allyloxycarbonylethyl or but-1-en-4-yloxycarbonylmethyl, in particular 2-allyloxycarbonylethyl;

C₃-C₄-alkynyloxy-C₁-C₄-alkyl: C₁-C₄-alkyl substituted by C₃-C₄-alkynyloxy such as propargyloxy, but-1-yn-3-yloxy, but-1-yn-4-yloxy, but-2-yn-1-yloxy, 1-methylprop-2-ynyloxy or 2-methylprop-2-ynyloxy, preferably propargyloxy, i.e., for example, propargyloxymethyl or 2-propargyloxyethyl, in particular 2-propargyloxyethyl;

C₃-C₄-alkynyloxycarbonyl-C₁-C₄-alkyl: C₁-C₄-alkyl substituted by C₃-C₄-alkynyloxycarbonyl such as propargyloxycarbonyl, but-1-yn-3-yloxycarbonyl, but-1-yn-4-yloxycarbonyl, but-2-yn-1-yloxycarbonyl, 1-methylprop-2-ynyloxycarbonyl or 2-methylprop-2-ynyloxycarbonyl, preferably propargyloxycarbonyl, i.e., for example, propargyloxycarbonylmethyl or 2-propargyloxycarbonylethyl, in particular 2-propargyloxycarbonylethyl;

C₃-C₈-cycloalkyl: cyclopropyl, cyclobutyl, cyclopentyl, cyclohexyl, cycloheptyl or cyclooctyl;

C₃-C₈-cycloalkyl-C₁-C₄-alkyl: C₁-C₄-alkyl which is substituted by C₃-C₈-cycloalkyl: e.g. cyclopropylmethyl, cyclobutylmethyl, cyclopentylmethyl, cyclohexylmethyl, cycloheptylmethyl, cyclooctylmethyl, 2-(cyclopropyl)ethyl, 2-(cyclobutyl)ethyl, 2-(cyclopentyl)ethyl, 2-(cyclohexyl)ethyl, 2-(cycloheptyl)ethyl, 2-(cyclooctyl)ethyl, 3-(cyclopropyl)propyl, 3-(cyclobutyl)propyl, 3-(cyclopentyl)propyl, 3-(cyclohexyl)propyl, 3-(cycloheptyl)propyl, 3-(cyclooctyl)propyl, 4-(cyclopropyl)butyl, 4-(cyclobutyl)butyl, 4-(cyclopentyl)butyl, 4-(cyclohexyl)butyl, 4-(cycloheptyl)butyl, 4-(cyclooctyl)butyl;

C₃-C₈-cycloalkoxy-C₁-C₄-alkyl: C₁-C₄-alkyl which is substituted by C₃-C₈-cycloalkoxy: e.g. cyclopropoxymethyl, cyclobutoxymethyl, cyclopentoxymethyl, cyclohexyloxymethyl, cycloheptyloxymethyl, cyclooctyloxymethyl, 2-(cyclopropyloxy)ethyl, 2-(cyclobutyloxy)ethyl, 2-(cyclopentyloxy)ethyl, 2-(cyclohexyloxy)ethyl, 2-(cycloheptyloxy)ethyl, 2-(cyclooctyloxy)ethyl, 3-(cyclopropyloxy)propyl, 3-(cyclobutyloxy)propyl, 3-(cyclopentyloxy)propyl, 3-(cyclohexyloxy)propyl, 3-(cycloheptyloxy)propyl, 3-(cyclooctyloxy)propyl, 4-(cyclopropyloxy)butyl, 4-(cyclobutyloxy)butyl, 4-(cyclopentyloxy)butyl, 4-(cyclohexyloxy)butyl, 4-(cycloheptyloxy)butyl, 4-(cyclooctyloxy)butyl;

C₃-C₈-cycloalkoxy is: cyclopropoxy, cyclobutoxy, cyclopentoxy, cyclohexoxy, cycloheptoxy or cyclooctoxy;

C₃-C₈-cycloalkyl-C₁-C₄-alkoxy is C₁-C₄-alkoxy which is substituted by C₃-C₈-cycloalkyl: e.g. cyclopropylmethoxy, cyclobutylmethoxy, cyclopentylmethoxy, cyclohexylmethoxy, cycloheptylmethoxy, cyclooctylmethoxy, 2-(cyclopropyl)ethoxy, 2-(cyclobutyl)ethoxy, 2-(cyclopentyl)ethoxy, 2-(cyclohexyl)ethoxy, 2-(cycloheptyl)ethoxy, 2-(cyclooctyl)ethoxy, 3-(cyclopropyl)propoxy, 3-(cyclobutyl)propoxy, 3-(cyclopentyl)propoxy, 3-(cyclohexyl)propoxy, 3-(cycloheptyl)propoxy, 3-(cyclooctyl)propoxy, 4-(cyclopropyl)butoxy, 4-(cyclobutyl)butoxy, 4-(cyclopentyl)butoxy, 4-(cyclohexyl)butoxy, 4-(cycloheptyl)butoxy, 4-(cyclooctyl)butoxy.

Examples of N-bonded 3-, 4-, 5-, 6- or 7-membered, saturated heterocyclic radicals are: aziridin-1-yl, azetidin-1-yl, pyrrolidin-1-yl, 1,3-oxazolidin-3-yl, 1,2-oxazolidin-2-yl, tetrahydropyrazol-1-yl, piperidin-1-yl, morpholin-4-yl, hexahydropyridazin-1-yl, hexahydropyrimidin-1-yl, piperazin-1-yl, hexahydro-1,3,5-triazin-1-yl, hexahydroazepin-1-yl, hexahydro-1,3-diazepin-1-yl and hexahydro-1,4-diazepin-1-yl.

With respect to the use of the compounds of the formula I as herbicides or for the desiccation and/or defoliation of plants, it has proven favorable if the variables X, R¹ and R² in formula I per se or in combination have the following meanings:

X is chlorine or bromine, in particular chlorine;

Y is fluorine;

R¹ is hydrogen or in particular methyl;

R² has one of the abovementioned meanings.

A preferred class of 2-phenyl-2H-pyridazin-3-ones according to the invention are those compounds of the general formula I in which Z is oxygen and R² is a group OR³ in which R³ has one of the abovementioned meanings which are preferably other than hydrogen. In the group OR³, R³ preferably has the following meanings:

R³ is C₁-C₄-alkyl, C₁-C₄-haloalkyl, amino-C₁-C₄-alkyl, C₁-C₄-cyanoalkyl, C₁-C₄-alkoxy-C₁-C₄-alkyl, C₁-C₄-alkylthio-C₁-C₄-alkyl, C₁-C₄-alkyl-C₁-C₄-sulfonyl-C₁-C₄-alkyl, C₁-C₄-alkylsulfinyl-C₁-C₄-alkyl, C₁-C₄-alkylamino-C₁-C₄-alkyl, di-C₁-C₄-alkylamino-C₁-C₄-alkyl, C₁-C₄-alkyloxycarbonyl-C₁-C₄-alkyl, C₃-C₄-alkenyl, C₃-C₄-alkynyl, C₃-C₄-alkenyloxy-C₁-C₄-alkyl, C₃-C₄-alkynyloxy-C₁-C₄-alkyl, C₃-C₄-alkenyloxycarbonyl-C₁-C₄-alkyl, C₃-C₄-alkynyloxycarbonyl-C₁-C₄-alkyl, C₃-C₈-cycloalkyl-C₁-C₄-alkyl,

in particular, C₁-C₄-alkyl, C₁-C₄-haloalkyl, C₁-C₄-cyanoalkyl, C₁-C₄-alkoxy-C₁-C₄-alkyl, C₁-C₄-alkylthio-C₁-C₄-alkyl, C₁-C₄-alkyloxycarbonyl-C₁-C₄-alkyl, C₃-C₄-alkenyl or C₃-C₄-alkynyl. Examples of preferred meanings of OR³ are the meanings indicated below in Table 1, lines 2 to 29.

If R² is a group of the general formula NR⁴R⁵, then the radicals preferably have the following meanings:

R⁴ is hydrogen, C₁-C₄-alkyl, C₁-C₄-alkoxy, C₁-C₄-haloalkyl, C₁-C₄-alkoxy-C₁-C₄-alkyl or C₁-C₄-alkyloxycarbonyl-C₁-C₄-alkyl and

R⁵ is hydrogen or C₁-C₄-alkyl, in particular hydrogen or methyl; or

R⁴ and R⁵, together with the nitrogen atom to which they are bonded, form a saturated 5- or 6-membered heterocyclic radical which optionally contains a further oxygen atom and/or an imino or C₁-C₄-alkylimino group as a ring member, for example a pyrrolidinyl, piperidinyl, piperazinyl or morpholinyl radical.

Examples of preferred groups of the general formula NR⁴R⁵ are the meanings indicated for R² in Table 1, lines 30 to 38.

Compounds of the general formula I in which Z is oxygen, X, Y and R¹ have the meanings mentioned beforehand, in particular the meanings mentioned as preferred, and in which R² is chlorine, C₁-C₄-alkoxy or OH are additionally of particular interest as intermediates for the preparation of other compounds of the formula I.

A further preferred class of 2-phenyl-2H-pyridazin-3-ones according to the invention are those compounds of the general formula I in which Z is a group NR⁶. In these, R⁶ is preferably C₁-C₄-alkoxy. R² is then a group OR⁷, in which R⁷ has the meanings mentioned beforehand and is in particular C₁-C₄-alkyl, C₁-C₄-haloalkyl, C₁-C₄-alkyloxycarbonyl-C₁-C₄-alkyl or C₁-C₄-haloalkyloxycarbonyl-C₁-C₄-alkyl.

Examples of particularly preferred compounds of the general formula I are the compounds of the general formula Ia indicated below (compounds I in which R¹ is CH₃, Y is fluorine and X is chlorine), in which Z and R² have the meanings indicated in line Nos 1 to 42 of Table 1. These compounds are also designated below as compounds Ia.1 to Ia.42.

TABLE 1

| | |
|---|---|
| | (Ia) |
| 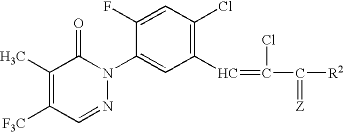 | |

| No. | Z | R² |
|---|---|---|
| 1 | O | OH |
| 2 | O | OCH₃ |
| 3 | O | OC₂H₅ |
| 4 | O | OCH₂CH₂CH₃ |
| 5 | O | O(CH₂)₃CH₃ |
| 6 | O | OCH(CH₃)₂ |
| 7 | O | OCH₂CH₂Cl |
| 8 | O | OCH₂CH₂OCH₃ |
| 9 | O | OCH₂CH₂OCH₂CH₃ |
| 10 | O | OCH₂CH₂CN |
| 11 | O | OCH₂CH₂SCH₃ |
| 12 | O | OCH₂CH₂SOCH₃ |
| 13 | O | OCH₂CH₂SO₂CH₃ |
| 14 | O | OCH₂CHNH₂ |
| 15 | O | OCH₂CH₂NCH₃ |
| 16 | O | OCH₂CH₂N(CH₃)₂ |
| 17 | O | OCH₂CO₂CH₃ |
| 18 | O | OCH₂CO₂CH₂CH₃ |
| 19 | O | OCH₂CO₂CH₂CH═CH₂ |
| 20 | O | OCH₂CO₂CH₂C≡CH |
| 21 | O | OCH(CH₃)CO₂CH₃ |
| 22 | O | OCH(CH₃)CO₂CH₂CH₃ |
| 23 | O | OCH(CH₃)CO₂CH₂CH═CH₂ |
| 24 | O | OCH(CH₃)CO₂CH₂C≡CH |
| 25 | O | OC(CH₃)₂CO₂CH₃ |
| 26 | O | OCH₂CH₂OCH₂CH₂Cl |
| 27 | O | OCH₂CH═CH₂ |
| 28 | O | OCH₂C≡CH |
| 29 | O | OCH₂cyclo—C₃H₅ |
| 30 | O | NH₂ |
| 31 | O | NHCH₃ |
| 32 | O | NH(CH₃)₂ |
| 33 | O | NHCH₂CH₂Cl |
| 34 | O | NHCH₂CO₂CH₃ |
| 35 | O | NHCH₂CO₂CH₂CH₃ |
| 36 | O | N(CH₃)CH₂CO₂CH₃ |
| 37 | O | N(CH₃)CH₂CO₂CH₂CH₃ |
| 38 | O | NHOCH₃ |
| 39 | NOCH₃ | OCH₂CO₂CH₃ |
| 40 | NOCH₃ | OCH₂CO₂CH₂Cl |
| 41 | NOCH₃ | OCH(CH₃)CO₂CH₃ |
| 42 | O | Cl |

Examples of particularly preferred compounds of the general formula I are furthermore the compounds of the general formula Ib indicated below, in which R¹ is H, Y is fluorine and X is Cl and in which Z and R² have the meanings indicated in line Nos 1 to 42 of Table 1 (compounds Ib.1 to Ib.42).

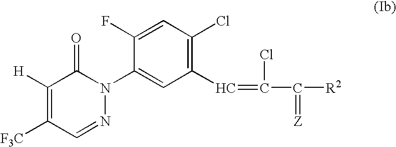

Examples of particularly preferred compounds of the general formula I are furthermore the compounds of the general formula Ic indicated below, in which R¹ is CH₃, Y is fluorine and X is Br and in which Z and R² have the meanings indicated in line Nos 1 to 42 of Table 1 (compounds Ic.1 to Ic.42).

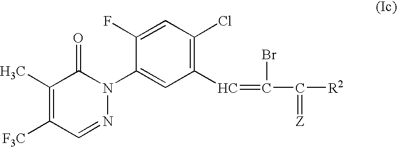

Further examples of particularly preferred compounds of the general formula I are the compounds of the general formula Id indicated below, in which R¹ is H, Y is fluorine and X is Br and in which Z and R² have the meanings indicated in line Nos 1 to 42 of Table 1 (compounds Id.1 to Id.42).

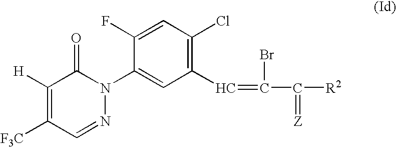

The compounds of the formula I can be prepared following WO 97/07104 or WO 99/52878, by first converting an aniline derivative of the formula III into a hydrazine of the formula IIa (compounds of the formula II where R^{a}=R^{b}=H), then condensing this with the ketone of the formula F₃C—C(O)—CHBr₂ (trifluorodibromoacetone) or another equivalent of the 2-oxo-3-trifluoropropanal to give a hydrazone of the general formula IIb (compounds II in which R^{a} and R^{b} form a group of the formula ═CH—C(O)—CF₃) and then reacting the compound IIb with a phosphorus compound of the formula IV in a Wittig reaction with subsequent ring closure to give a pyridazinone of the formula I. In formula IV, the radical R as a rule is a C₁-C₄-alkyl group, e.g. ethyl. In Scheme 1, the variables X, Y, Z and R² have the meanings mentioned beforehand. In a preferred embodiment of the process shown in Scheme 1, Z is O and R² is a group OR³ where R³≠H and in particular is C₁-C₄-alkoxy.

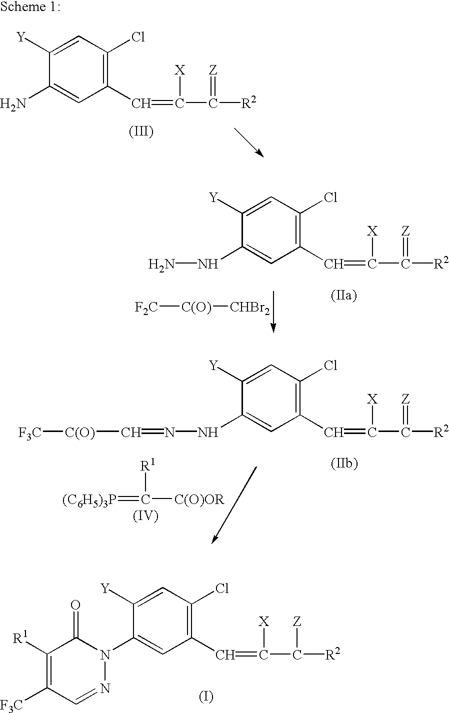

The 3-aminocinnamic acid compounds of the formula III are in some cases known from the literature, for example from EP-A 240 659, EP-A 300 387 and DE-A 39 04 082, or can be prepared analogously to the methods described there.

The conversion of the 3-aminocinnamic acid compound III into the corresponding hydrazine compound IIa can be carried out according to the methods known for the conversion of aniline compounds into aromatic hydrazines (see, for example, Houben-Weyl, Volume EI, Nitrogen Compounds I, Georg Thieme Verlag 1967). Typical processes include the diazotization of the amino group in III, for example by reaction of III with nitrite salts such as sodium nitrite in the presence of mineral acids, e.g. by reaction in concentrated hydrochloric acid, and subsequent reduction of the diazonium compounds obtained here, e.g. with tin(II) chloride under acidic reaction conditions.

The preparation of the hydrazones of the formula IIb is possible, for example, by reaction of the hydrazine IIa with a derivative of 2-oxo-3-trifluoropropanal such as trifluorodibromoacetone according to the methods described in WO 97/07104 and WO 99/52878. Derivatives of 2-oxo-3-trifluoropropanal such as trifluorodibromoacetone (CAS No. 431-67-4) are in some cases commercially obtainable or can be prepared by processes known from the literature.

The preparation of I by reaction of compound IIb with a compound IV is carried out in a Wittig or Wittig-Horner reaction under the reaction conditions customary for this purpose, such as are described, for example, in WO 97/07104 or DE-A 197 54 348. The phosphorus compounds of the formula IV needed are known from the literature or can be prepared according to known literature processes. In some cases, they are commercially obtainable, e.g. (1-ethoxycarbonylethylidene)triphenylphosphorane (CAS No. 5717-37-3).

It has furthermore been found that compounds of the formula I in which R² is a radical OR³ having the meanings mentioned beforehand for R³, for example C₁-C₄-alkyl, can be converted into other compounds of the general formula I according to Scheme 2 below. In Scheme 2, R^{2a} in formula I′ is thus OR³ having the meanings mentioned beforehand for R³, e.g. where R³=C₁-C₄-alkyl. Compounds I′ where R^{2a}=OR³=O—C₁-C₄-alkyl are also designated below as lower alkyl esters I.

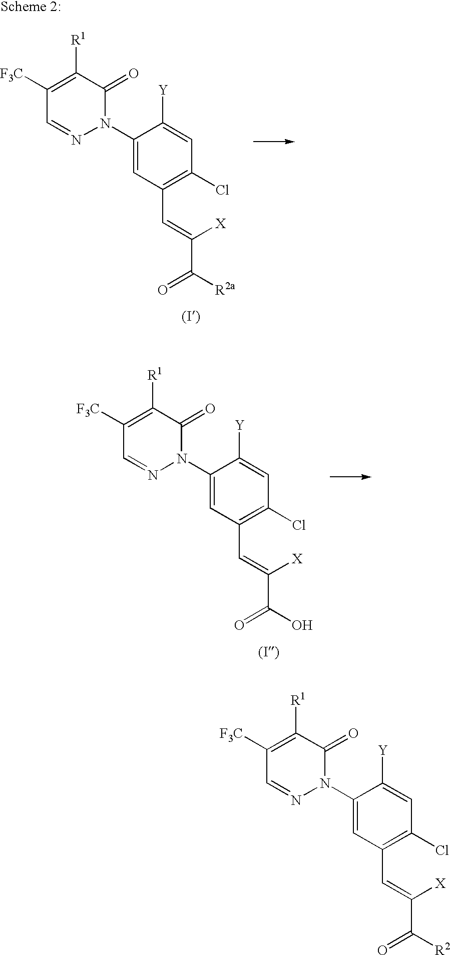

For this, according to Scheme 2 compounds of the formula I′ are hydrolyzed to the free acid of the formula I″ (R²=OH) and, preferably after activation, for example to the acid chloride (compounds of the formula I where R²=Cl), reacted again with a further alcohol HOR³, an amine HNR⁴R⁵ or a hydroxylamine ether H₂NR⁶ to give a compound I in the form of an ester derivative (R²=OR³) or an amide derivative (R²=NR⁴R⁵ or NHR⁶). Here and below, the variables R³-R⁶ have the meanings mentioned beforehand.

For the hydrolysis, the ester I′, for example, preferably a lower alkyl ester I′ (R^{2a}=O—C₁-C₄-alkyl), is introduced in a mixture of acid, water and, if appropriate, a solvent. The hydrolysis is optionally carried out with warming.

Suitable acids are mineral acids such as hydrochloric acid, hydrobromic acid, sulfuric acid or phosphoric acid and also trifluoroacetic acid, which as a rule are employed as aqueous acids (concentration, for example 5 to 98% by weight). In addition to water, preferred solvents are also organic carboxylic acids such as acetic acid.

The reaction is customarily carried out in a mixture of aqueous acid and solvent, where the volume ratio of aqueous acid to solvent can as a rule be from 1:99 to 99:1, and is preferably in the range from 1:4 to 4:1. The reaction temperatures necessary for the hydrolysis are as a rule in the range from 20 to 160° C., but preferably from 20 to 120° C.

All customary activation methods are suitable for the activation of the acid I″, e.g. conversion into the acid chloride, activation with carbodiimides such as dicyclohexylcarbodiimide or, in the case of reaction of I″ with alcohols HOR³, classical proton catalysis with mineral acids such as sulfuric acid.

Preferably, the activation is carried out by reaction of I″ to give the acid chloride of the formula I (R²=Cl), which is typically carried out using thionyl chloride, oxalyl chloride or PCl₃ or PCl₅ as a chlorinating agent. Suitable solvents here are inert solvents such as methylene chloride, chloroform, dichloroethane or toluene. However, the reaction can also be carried out in the chlorinating agent itself as a solvent or in the melt. Depending on the chlorinating agent, the reaction is carried out with 1 to 5 equivalents of the chlorinating agent and at temperatures between −78° C. and 150° C.

The further reaction of the acid chloride I (R²=Cl) with the alcohol HOR³ or the amine HNR⁴R⁵ or the hydroxylamine ether H₂NR⁶ is as a rule likewise carried out in an inert solvent such as methylene chloride, chloroform, dichloroethane or toluene. Preferably, 1 to 5 equivalents of the alcohol HOR³ or amine HNR⁴R⁵ or H₂NR⁶, based on the acid chloride, are employed for this purpose. If appropriate, the reaction is carried out with addition of preferably 1 to 5 equivalents of an auxiliary base, e.g. a trialkylamine such as triethylamine or pyridine. The reaction is preferably carried out at temperatures in the range from 0° C. to 100° C.

A further advantageous embodiment of this reaction consists in introducing the acid I″ in the alcohol HOR³ intended for the esterification or a mixture of this alcohol with one of the abovementioned solvents and reacting it in situ with thionyl chloride.

A further advantageous embodiment of the reaction consists in introducing the acid I″ in the alcohol HOR³ intended for the esterification or a mixture of this alcohol with one of the abovementioned solvents as solvent and reacting it in the presence of a catalytic amount of a mineral acid such as sulfuric acid. In this case, the amount of mineral acid can be between 1 and 100 mol %, based on the acid I (R²=OH).

A further derivatization is the conversion of the esters of the formula I′, preferably of the lower alkyl esters I′ (R^{2a}=O—C₁-C₄-alkyl) into the hydroxamic acid esters of I (Z=O and R²=NHR⁶), which for their part can be converted by alkylation using an alkylating agent of the formula X—R⁷, in which X is a nucleophilically displaceable leaving group and R⁷ has the meanings indicated beforehand, into the iminoesters of the formula I where Z=NR⁶ and R²=OR⁷. R⁶ has the meanings indicated beforehand.

The hydroxamic acid esters can be prepared starting from the carboxylic acids of the formula I″ in the manner described above by successive activation of the carboxylic acid and subsequent reaction with the hydroxylamine ether H₂NR⁶. For the preparation of the compounds of the formula I where Z=NR⁶ and R²=OR⁷ (iminoesters), the hydroxamic acid ester I, if appropriate in the presence of a base, is reacted with an alkylating agent of the formula X—R⁷ (or [(R⁷)₂OR⁷]⁺Y⁻), an O-alkylation of the carbonyl oxygen in I taking place.

Suitable alkylating reagents are trialkyloxonium salts [(R⁷)₂OR⁷]⁺Y⁻, in which R⁷ has the meanings mentioned beforehand and Y⁻ is a nonnucleophilic anion such as tetrafluoroborate or perchlorate, and aliphatic halides R⁷—X, in which X is a halogen atom and R⁷ has the meanings mentioned beforehand. Aliphatic bromides R⁷—Br, such as ethyl bromide, allyl bromide, propargyl bromide and α-bromocarboxylic acids and their esters such as α-bromoacetic acid and α-bromopropionic acid and their esters, for example their methyl and ethyl esters, are particularly suitable.

As a rule, the reaction is carried out in a solvent. Suitable solvents, depending on the alkylating agent, are polar solvents such as dimethylformamide (DMF), N-methylpyrrolidone (NMP), THF, acetone, acetonitrile or else CH₂Cl₂; acetone is particularly suitable.

Bases which can be employed are, for example, the hydroxides and carbonates of the alkali metals and alkaline earth metals. The carbonates of sodium or potassium are preferred.

As a rule, the reaction is carried out in a temperature range from 0 up to the boiling temperature of the respective solvent. In a preferred embodiment, the reaction is carried out at 0 to 50° C., but very particularly preferably without the supply or removal of heat.

The compounds I and their agriculturally utilizable salts are suitable—both as isomer mixtures and in the form of the pure isomers—as herbicides. The compounds I or herbicidal compositions comprising their salts control vegetation very well on non-crop areas, particularly at high application rates. In crops such as wheat, rice, corn, soybeans and cotton, they act against weeds and weed grasses without noticeably damaging the crop plants. This effect occurs especially at low application rates.

Depending on the particular application method, the compounds I or compositions comprising them can additionally be employed for controlling undesired plants in a further number of crop plants. The following crops, for example, are suitable:

Allium cepa, Ananas comosus, Arachis hypogaea, Asparagus officinalis, Beta vulgaris spec. altissima, Beta vulgaris spec. rapa, Brassica napus var. napus, Brassica napus var. napobrassica, Brassica rapa var. silvestris, Camellia sinensis, Carthamus tinctorius, Carya illinoinensis, Citrus limon, Citrus sinensis, Coffea arabica (Coffea canephora, Coffea liberica), Cucumis sativus, Cynodon dactylon, Daucus carota, Elaeis guineensis, Fragaria vesca, Glycine max, Gossypium hirsutum, (Gossypium arboreum, Gossypium herbaceum, Gossypium vitifolium), Helianthus annuus, Hevea brasiliensis, Hordeum vulgare, Humulus lupulus, Ipomoea batatas, Juglans regia, Lens culinaris, Linum usitatissimum, Lycopersicon lycopersicum, Malus spec., Manihot esculenta, Medicago sativa, Musa spec., Nicotiana tabacum (N. rustica), Olea europaea, Oryza sativa, Phaseolus lunatus, Phaseolus vulgaris, Picea abies, Pinus spec., Pisum sativum, Prunus avium, Prunus persica, Pyrus communis, Ribes sylvestre, Ricinus communis, Saccharum officinarum, Secale cereale, Solanum tuberosum, Sorghum bicolor (S. vulgare), Theobroma cacao, Trifolium pratense, Triticum aestivum, Triticum durum, Vicia faba, Vitis vinifera, Zea mays.

Moreover, the compounds I can also be used in crops which have been made tolerant to the action of herbicides by means of breeding, including genetic engineering methods.

In addition, the 2-phenyl-2H-pyridazin-3-ones of the general formula I according to the invention and their agriculturally utilizable salts are also suitable for the desiccation and/or defoliation of plants.

As desiccants, they are suitable, in particular, for drying out the above-ground parts of crop plants such as potatoes, rapeseed, sunflower and soybeans. Completely mechanical harvesting of these important crop plants is made possible in this way.

Also of economic interest is the time-controlled fall of fruit or the reduction in their firmness of attachment to the plant, for example in the case of citrus fruits, olives and other types of pomes, drupes and indehiscent fruit, since by this means the harvesting of this fruit is facilitated. The fall is based on the formation of abscission tissue between the fruit, leaf and sprout part of the plants and is promoted by the 2-phenyl-2H-pyridazin-3-ones of the general formula I according to the invention and their salts. The use of the 2-phenyl-2H-pyridazin-3-ones of the general formula I according to the invention and their agriculturally utilizable salts thus allows controlled fall of fruits and controlled defoliation of the crop plants such as cotton and thus makes possible facilitation of harvesting in crop plants of this type. Controlled defoliation is particularly also of interest in the case of useful plants such as cotton. By means of the shortening of the time interval in which the individual cotton plants become ripe, increased quality of the harvested fiber material is achieved.

The compounds of the formula I according to the invention or the herbicidal compositions comprising them can be applied by spraying, atomizing, dusting, broadcasting, watering or treatment of the seed or mixing with the seed in the form of directly sprayable aqueous solutions, powders, suspensions, also high-percentage aqueous, oily or other suspensions or dispersions, emulsions, oil dispersions, pastes, dusting compositions, broadcasting compositions or granules. The use forms depend on the intended use; in each case they should if possible guarantee the finest dispersion of the active compounds according to the invention. The compositions according to the invention contain a herbicidally active amount of at least one compound of the general formula I or an agriculturally utilizable salt of I and the auxiliaries customary for the formulation of crop protection compositions.

Suitable inert additives are essentially: mineral oil fractions of medium to high boiling point, such as kerosene or diesel oil, furthermore coal tar oils and oils of vegetable or animal origin, aliphatic, cyclic and aromatic hydrocarbons, e.g. paraffin, tetrahydronaphthalene, alkylated naphthalenes or their derivatives, alkylated benzenes or their derivatives, alcohols such as methanol, ethanol, propanol, butanol, cyclohexanol, ketones such as cyclohexanone or strongly polar solvents, e.g. amines such as N-methylpyrrolidone or water.

Aqueous application forms can be prepared from emulsion concentrates, suspensions, pastes, wettable powders or water-dispersible granules by addition of water. For the preparation of emulsions, pastes or oil dispersions, the compounds I can be homogenized as such or dissolved in an oil or solvent, by means of wetting agents, adhesives, dispersants or emulsifiers. However, concentrates consisting of active substance, wetting agent, adhesive, dispersant or emulsifier and possibly solvent or oil can also be prepared, which are suitable for dilution with water.

Suitable surface-active substances are the alkali metal, alkaline earth metal and ammonium salts of aromatic sulfonic acids, e.g. lignosulfonic, phenolsulfonic, naphthalenesulfonic and dibutylnaphthalenesulfonic acid, and also of fatty acids, alkyl-and alkylarylsulfonates, alkylsulfates, lauryl ether sulfates and fatty alcohol sulfates, and also salts of sulfated hexa-, hepta-and octadecanols and of fatty alcohol glycol ethers, condensation products of sulfonated naphthalene and its derivatives with formaldehyde, condensation products of naphthalene or of naphthalenesulfonic acids with phenol and formaldehyde, polyoxyethyleneoctylphenyl ether, ethoxylated isooctyl-, octyl- or nonylphenol, alkylphenyl or tributylphenyl polyglycol ethers, alkylaryl polyether alcohols, isotridecyl alcohol, fatty alcohol-ethylene oxide condensates, ethoxylated castor oil, polyoxyethylene alkyl ethers or polyoxypropylene alkyl ethers, lauryl alcohol polyglycol ether acetate, sorbitol esters, lignin-sulfite waste liquors or methylcellulose.

Powders, broadcasting compositions and dusting compositions can be prepared by mixing or joint grinding of the active substances with a solid carrier.

Granules, e.g. coated, impregnated and homogeneous granules, can be prepared by binding the active compounds to solid carriers. Solid carriers are mineral earths such as silicic acids, silica gels, silicates, talc, kaolin, limestone, lime, chalk, bole, loess, clay, dolomite, diatomaceous earths, calcium sulfate and magnesium sulfate, magnesium oxide, ground synthetic materials, fertilizers, such as ammonium sulfate, ammonium phosphate, ammonium nitrate, ureas and vegetable products such as grain flour, tree bark meal, wood meal and nutshell meal, cellulose powder or other solid carriers.

The concentrations of the active compounds I in the ready-to-use preparations can be varied within wide ranges. The formulations in general contain 0.001 to 98% by weight, preferably 0.01 to 95% by weight, of at least one active compound. The active compounds are employed here in a purity of from 90% to 100%, preferably 95% to 100% (according to NMR spectrum).

The compounds I according to the invention can be formulated, for example, as follows:

I 20 parts by weight of the compound No. Ia.3 are dissolved in a mixture which consists of 80 parts by weight of alkylated benzene, 10 parts by weight of the addition product of 8 to 10 mol of ethylene oxide to 1 mol of oleic acid N-monoethanolamide, 5 parts by weight of calcium salt of dodecylbenzenesulfonic acid and 5 parts by weight of the addition product of 40 mol of ethylene oxide to 1 mol of castor oil. By pouring out and finely dispersing the solution in 100 000 parts by weight of water, an aqueous dispersion is obtained which contains 0.02% by weight of the active compound.

II 20 parts by weight of the compound No. Ia.2 are dissolved in a mixture which consists of 40 parts by weight of cyclohexanone, 30 parts by weight of isobutanol, 20 parts by weight of the addition product of 7 mol of ethylene oxide to 1 mol of isooctylphenol and 10 parts by weight of the addition product of 40 mol of ethylene oxide to 1 mol of castor oil. By pouring the solution into 100 000 parts by weight of water and finely dispersing it, an aqueous dispersion is obtained which contains 0.02% by weight of the active compound.

III 20 parts by weight of the active compound No. Ia.17 are dissolved in a mixture which consists of 25 parts by weight of cyclohexanone, 65 parts by weight of a mineral oil fraction of boiling point 210 to 280° C. and 10 parts by weight of the addition product of 40 mol of ethylene oxide to 1 mol of castor oil. By pouring the solution into 100 000 parts by weight of water and finely dispersing it, an aqueous dispersion is obtained which contains 0.02% by weight of the active compound.

IV 20 parts by weight of the active compound No. Ia.39 are well mixed with 3 parts by weight of the sodium salt of diisobutylnaphthalenesulfonic acid, 17 parts by weight of the sodium salt of a lignosulfonic acid from a sulfite waste liquor and 60 parts by weight of powdered silica gel and ground in a hammer mill. By finely dispersing the mixture in 20 000 parts by weight of water, a spray liquor is obtained which contains 0.1% by weight of the active compound.

V 3 parts by weight of the active compound No. Ia.3 are mixed with 97 parts by weight of finely divided kaolin. A dusting composition which contains 3% by weight of the active compound is obtained in this way.

VI 20 parts by weight of the active compound No. Ia.2 are intimately mixed with 2 parts by weight of calcium salt of dodecylbenzenesulfonic acid, 8 parts by weight of fatty alcohol polyglycol ether, 2 parts by weight of sodium salt of a phenol/urea/formaldehyde condensate and 68 parts by weight of a paraffinic mineral oil. A stable oily dispersion is obtained.

VII 1 part by weight of the compound No. Ia.17 is dissolved in a mixture which consists of 70 parts by weight of cyclohexanone, 20 parts by weight of ethoxylated isooctylphenol and 10 parts by weight of ethoxylated castor oil. A stable emulsion concentrate is obtained.

VIII 1 part by weight of the compound No. Ia.39 is dissolved in a mixture which consists of 80 parts by weight of cyclohexanone and 20 parts by weight of Wettol® EM 31 (nonionic emulsifier based on ethoxylated castor oil). A stable emulsion concentrate is obtained.

The herbicidal compositions or the active compounds which contain the 2-phenyl-2H-pyridazin-3-ones of the general formula I and/or their salts can be applied preemergence, postemergence or together with the seed of a crop plant. There is also the possibility of applying the herbicidal compositions or active compounds by applying seed of a crop plant pretreated with the herbicidal compositions or active compounds. If the active compounds are less tolerable to certain crop plants, application techniques can be used in which the herbicidal compositions are sprayed with the aid of spray equipment such that the leaves of the sensitive crop plants are not affected if possible, while the active compounds reach the leaves of undesired plants growing thereunder or the uncovered soil surface (post-directed, lay-by).

Depending on the aim of control, time of year, target plants and stage of growth, the application rates of active compound are 0.001 to 3.0, preferably 0.01 to 1.0, kg/ha of active substance (a.s.).

To widen the spectrum of action and to achieve synergistic effects, the compounds of the general formula I according to the invention can be mixed and applied together with numerous representatives of other herbicidal or growth-regulating active compound groups. For example, suitable mixture components are 1,2,4-thiadiazoles, 1,3,4-thiadiazoles, amides, aminophosphoric acids and their derivatives, aminotriazoles, anilides, (het)aryloxyalkanoic acids and their derivatives, benzoic acid and its derivatives, benzothiadiazinones, 2-aroyl-1,3-cyclohexanediones, hetaryl aryl ketones, benzylisoxazolidinones, meta-CF₃-phenyl derivatives, carbamates, quinolinecarboxylic acid and its derivatives, chloroacetanilides, cyclohexane-1,3-dione derivatives, diazines, dichloropropionic acid and its derivtives, dihydrobenzofurans, dihydrofuran-3-ones, dinitroanilines, dinitrophenols, diphenyl ethers, dipyridyls, halocarboxylic acids and their derivatives, ureas, 3-phenyluracils, imidazoles, imidazolinones, N-phenyl-3,4,5,6-tetrahydrophthalimides, oxadiazoles, oxiranes, phenols, aryloxy- or hetaryloxyphenoxypropionic acid esters, phenylacetic acid and its derivatives, phenylpropionic acid and its derivatives, pyrazoles, phenylpyrazoles, pyridazines, pyridinecarboxylic acid and its derivatives, pyrimidyl ethers, sulfonamides, sulfonylureas, triazines, triazinones, triazolinones, triazolecarboxamides and uracils.

Moreover, it may be useful to apply the compounds I alone or jointly, in combination with other herbicides additionally mixed with further crop protection agents, for example with agents for controlling pests or phytopathogenic fungi or bacteria. Furthermore of interest is the miscibility with mineral salt solutions, which can be employed for the abolishment of nutritional and trace element deficiencies. Nonphytotoxic oils and oil concentrates can also be added.

The following examples are intended to illustrate the invention:

The following abbreviations were used:

s=singlet

d=doublet

dd=doublet of doublet

t=triplet

q=quartet

m=multiplet

br=broad signal.

All signals are indicated as the chemical shift in ppm against tetramethylsilane (TMS). Moreover, the number of hydrogen atoms to be assigned to the signal is indicated in each case.

### I PREPARATION EXAMPLES

### Example 1

### 2-[4-Chloro-2-fluoro-5-(2-ethoxycarbonyl-2-chloro-ethen-1-yl)phenyl]-4-methyl-5-trifluormethyl-2H-pyridazin-3-one

1.1 4-Chloro-2-fluoro-5-(2-ethoxycarbonyl-2-chloroethen-1-yl)-phenylhydrazine (compound IIa.1)

15.0 g (0.09 mol) of 4-chloro-2-fluoro-5-(2-ethoxycarbonyl-2-chloroethen-1-yl)aniline were initially introduced in 140 ml of concentrated hydrochloric acid and treated dropwise at 0 to 5° C. with 4.1 g (0.09 mol) of sodium nitrite, dissolved in 15 ml of water. After a further 1 h at 0 to 5° C., the reaction mixture was added to a solution of 30.4 g (0.13 mol) of tin(II) chloride hydrate in 100 ml of concentrated hydrochloric acid and the mixture was stirred for 3 h at 0 to 5° C. The mixture thus obtained was poured into ice water, adjusted to pH 12 using 50% strength by weight aqueous sodium hydroxide solution and the deposited product was filtered off. After washing and drying, 12.5 g of 4-chloro-2-fluoro-5-(2-ethoxycarbonyl-2-chloroethen-1-yl)-phenylhydrazine (compound IIa.1) remained.

¹H NMR* (D₆-DMSO): 8.1 (s, 1H), 7.8 (d, 1 H), 7.0 (d, 1 H), 7.1 (s, 1 H), 5.9 (br. s, 1 H), 4.4 (q, 2 H), 4.8-4.2 (br, 2 H), 1.3 (t, 3 H)

1.2 4-Chloro-2-fluoro-5-(2′-ethoxycarbonyl-2′-chloroethen-1-yl)-phenylhydrazone of 3,3,3,-trifluoro-2-oxopropanal (compound IIb.1)

8.8 g (0.106 mol) of sodium acetate were dissolved in 110 ml of water and 12.7 g (0.047 mol) of trifluorodibromoacetone were added thereto. After 20 min at 70° C., the mixture was cooled to room temperature and a solution of 12.5 g (0.043 mol) of the hydrazine IIa.1 in 110 ml of diethyl ether was added thereto. The mixture was stirred overnight at room temperature, the organic phase was separated off and the aqueous phase was extracted with 100 ml of diethyl ether. Evaporation of the combined organic phases afforded the title compound IIb.1 in approximately 90% purity (19.1 g).

1.3 2-[4-Chloro-2-fluoro-5-(2-ethoxycarbonyl-2-chloroethen-1-yl)-phenyl]-4-methyl-5-trifluoromethyl-2H-pyridazin-3-one (compound Ia.3)

19.0 g (about 0.047 mol) of the crude product from Example 1.2 (hydrazone IIb.1) were dissolved in 160 ml of tetrahydrofuran (THF). 17.4 g (0.047 mol) of (1-ethoxycarbonylethylidene)triphenylphosphorane were added thereto. After 3 h at reflux, the mixture was washed with DMF/H₂O, the organic phase was concentrated and the solid residue was chromatographed (cyclohexane/ethyl acetate). The compound Ia.3 was obtained in a yield of 9.8 g. Melting point 131-133° C.

¹H NMR (CDCl₃): 8.2 (d, 1H), 8.1 (s, 1H), 8.0 (s, 1 H), 7.4 (d, 1 H), 4.4 (q, 2 H), 2.4 (t, 3 H), 1.4 (t, 3 H)

### Example 2

### 2-[4-Chloro-2-fluoro-5-(2-hydroxycarbonyl-2-chloro-ethen-1-yl)phenyl]-4-methyl-5-trifluoromethyl-2H-pyridazin-3-one (compound Ia.1)

9.4 g (0.02 mol) of the pyridazinone Ia.3 from Example 1 were stirred at 80° C. for 4 h in 140 ml of a 1:1 (v/v) mixture of concentrated hydrochloric acid and glacial acetic acid and cooled. The title compound (acid Ia.1) was deposited here as a solid, which was filtered off with suction. Moreover, the mother liquor was extracted with toluene and concentrated to dryness in vacuo. A total of 7.6 g of the compound Ia.1 was thus obtained. Melting point 174-178° C.

### Example 3

### 2-[4-Chloro-2-fluoro-5-(2-chlorocarbonyl-2-chloro-ethen-1-yl)phenyl]-4-methyl-5-trifluoromethyl-2H-pyridazin-3-one (compound Ia.42)

7.5 g (0.018 mol) of acid Ia.1 from Example 2 were heated to reflux in 90 ml of thionyl chloride for 3 h and then the thionyl chloride was removed in vacuo. The acid chloride Ia.42 obtained in the reaction was directly reacted further.

### Example 4

### 2-[4-Chloro-2-fluoro-5-(2-(N-methoxy)aminocarbonyl-2-chloroethen-1-yl)phenyl]-4-methyl-5-trifluoro-methyl-2H-pyridazin-3-one (compound Ia.38)

1.5 g (0.018 mol) of O-methylhydroxylamine hydrochloride were dissolved in 60 ml of dichloromethane and treated with 4.6 g (0.046 mol) of triethylamine and a catalytic amount of 4-dimethylaminopyridine. A solution of the acid chloride (compound Ia.42) obtained according to Example 3 in 60 ml of methylene chloride was added dropwise to this solution. The mixture was stirred until a thin-layer chromatogram (silica gel, cylcohexane/ethyl acetate) no longer showed any further change. The solution was concentrated in vacuo and the residue was chromatographed on silica gel (cyclohexane/ethyl acetate). The compound Ia.38 was thus obtained in a yield of 3.4 g.

### Example 5

### 4-(4-Methyl-5-trifluoromethyl-2H-pyridazin-3-on-2-yl)-1-chloro-3-fluoro-6-[2-chloro-3-methoxyimino-3-(methoxycarbonylmethyloxy)-1-propen-1-yl]benzene (compound Ia.39)

0.4 g (0.09 mol) of the compound Ia.38 obtained according to Example 4 were treated with 0.15 g (1.1 mmol) of potassium carbonate and 0.17 g (1.1 mmol) of methyl bromoacetate in 20 ml of acetone. The mixture was stirred at room temperature for 48 h, the solvent was removed in vacuo and the residue was partitioned between water and dichloromethane. The organic phase was separated off and the solvent was evaporated in vacuo. After chromatography of the residue on silica gel, 0.3 g of the compound Ia.39 was obtained. Melting point 75-76° C.

¹H NMR (D₆-DMSO): 8.3 (s, 1H), 8.1 (d, 1H), 7.9 (d, 1 H), 7.7 (s, 1 H), 5.0 (s, 1 H), 4.8 (s, 1H), 4.7 (s, 3H), 2.4 (t, 3H)

### Example 6

### 2-[4-Chloro-2-fluoro-5-(2-methoxycarbonyl-2-chloro-ethen-1-yl)phenyl]-4-methyl-5-trifluoromethyl-2H-pyridazin-3-one (compound Ia.2)

By reaction of the acid chloride Ia.42 from Example 3 with methanol in the presence of an auxiliary base, it was possible to obtain the compound Ia.2. Melting point 142-145° C.

### Example 7

### 2-[4-Chloro-2-fluoro-5-{2-(methoxycarbonylmethyloxy-carbonyl)-2-chloroethenyl}phenyl]-4-methyl-5-trifluoromethyl-2H-pyridazin-3-one (compound Ia.17)

By reaction of the acid chloride with methyl hydroxyacetate in the presence of an auxiliary base, it was possible to obtain the compound Ia.17. Melting point 88-91° C.

### Example 8

Analogously to Example 4, by reaction of the acid chloride Ia.42 with methyl glycinate the compound Ia.34 was prepared.

¹H NMR (CDCl₃) δ: 8.2 (br.s, 1H), 8.1-7.9 (m, 2H), 7.4 (d, 1H), 7.3 (d, 1H), 4.2 ps. (d 2H), 3.8 (s 3H)

### Example 9

Analogously to Example 6, the acid chloride Ia.42 was reacted with 2-chloroethanol, the compound Ia.7 being obtained.

¹H NMR (CDCl₃) δ: 8.2 (m, 1H), 8.00 (S, 1H), 7.4 (d, 1H), 4.6 (m, 2H), 3.8 (m, 2H)

The compounds of Examples 1 to 9 are compiled in Table 2. The compounds of Examples 10 to 18 below are shown in Table 2a. All compounds indicated in Tables 2 and 2a are present to at least 95% as Z isomers.

### Example 10

### 2-[4-Chloro-2-fluoro-5-(2-ethoxycarbonyl-2-chloro-ethen-1-yl)phenyl]-5-trifluoromethyl-2H-pyridazin-3-one (compound Ib.3)

Analogously to Example 1.3, the crude product obtained in Example 1.2 was reacted with (1-ethoxycarbonylmethylidene)-triphenylphosporane. The title compound Ib.3 had a melting point of 139 to 140° C.

### Example 11

### 2-[4-Chloro-2-fluoro-5-(2-chlorocarbonyl-2-chloro-ethen-1-yl)phenyl]-5-trifluoromethylpyridazin-3-one (compound Ib.42)

Analogously to Example 2 and Example 3, the acid chloride Ib.42 was prepared starting from the ester Ib.3.

### Examples 12 to 18

Analogously to Example 6 or 4, the pyridazinone compounds indicated in Table 2a were prepared starting from the acid chloride Ib.42.

TABLE 2

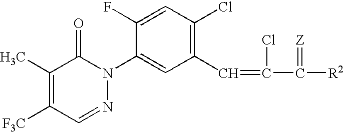

| Example | Compound¹⁾ | Z | R² |
|---|---|---|---|
| 1 | Ia.3 | O | OC₂H₅ |
| 2 | Ia.1 | O | OH |
| 3 | Ia.42 | O | Cl |
| 4 | Ia.38 | O | NHOCH₃ |
| 5 | Ia.39 | NOCH₃ | OCH₂—CO₂—CH₃ |
| 6 | Ia.2 | O | OCH₃ |
| 7 | Ia.17 | O | OCH₂CO₂CH₃ |
| 8 | Ia.34 | O | NH—CH₂—CO₂—CH₃ |
| 9 | Ia.7 | O | OCH₂CH₂—Cl |

¹⁾see Table 1

TABLE 2a

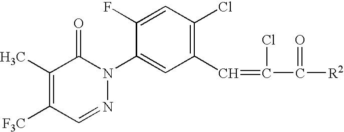

| Ex- | | | | |
|---|---|---|---|---|
| am- | | | | m.p.¹⁾ |
| ple | No. | R² | ¹H-NMR (CDCl₃) | [° C.] |
| 10 | Ib.3 | OC₂H₅ | — | 139-140 |
| 12 | Ib.30 | NH₂ | 8.2(d, 1H), 8.1(m, | 128-129 |
| | | | 1H), 8.0(m, 1H), | |
| | | | 7.4(d, 1H), 7.3(d, 1H), | |
| | | | 6.7(br. s 1H), 6.1(br. | |
| | | | s 1H) | |
| 13 | Ib.36 | N(CH₃)CH₂CO₂CH₃ | 8.1(m 1H), 7.4-7.3(m, | |
| | | | 2H), 7.2-7.0(m, 1H), | |
| | | | 3.8(s, 3H), 3.2-3.0(br. | |
| | | | 3H) | |
| 14 | Ib.34 | NHCH₂CO₂CH₃ | 8.2(d, 1H), 8.1(m, | 113-117 |
| | | | 2H), 8.0(d, 1H), 7.4(d, | |
| | | | 1H), 7.3-7.0(m, 2H), | |
| | | | 4.2(d, 2H), 3.8(s, 3H) | |
| 15 | Ib.17 | OCH₂CO₂CH₃ | 8.2(m, 2H), 8.1(d, | 122-123 |
| | | | 1H), 7.4(d, 1H), | |
| | | | 7.3(br. s, 1H), 4.8(s, | |
| | | | 2H), 3.8(s, 3H) | |
| 16 | Ib.7 | OCH₂CH₂Cl | 8.2(m, 2H), 8.1(m, | 94 |
| | | | 1H), 7.4(d, 1H), 7.3(d, | |
| | | | 1H), 4.6(m, 2H), | |
| | | | 3.8(m, 2H) | |
| 17 | Ib.27 | OCH₂CH═CH₂ | 8.2(d, 1H), 8.1(m, | 117-118 |
| | | | 2H), 7.4(d, 1H), | |
| | | | 7.3(br. s, 1H), 6.0(m, | |
| | | | 1H), 5.4(dd, 1H), | |
| | | | 4.8(m, 2H) | |
| 18 | Ib.2 | OCH₃ | 8.2(m, 1H), 8.1(m, | 121-123 |
| | | | 2H), 7.4(d, 1H), 7.3 | |
| | | | (d, 1H), 4.9 (s, 3H) | |

¹⁾m.p. = melting point

### II USE EXAMPLES

### II.1 Herbicidal Action

The herbicidal action of the 2-phenylpyridazin-3-one compounds of the formula I could be shown by means of greenhouse experiments:

The cultivation containers used were plastic pots containing loamy sand with approximately 3.0% of humus as a substrate. The seeds of the test plants were sown separately according to species.

In the case of preemergence treatment, the active compounds suspended or emulsified in water were applied directly after sowing by means of finely dispersing nozzles. The containers were lightly watered in order to promote germination and growth, and then covered with transparent plastic hoods until the plants had taken root. This covering brings about uniform germination of the test plants if this has not been adversely affected by the active compounds.

For the purpose of postemergence treatment, the test plants were first raised up to a growth height of 3 to 15 cm, depending on growth form, and then treated with the active compounds suspended or emulsified in water. For this, the test plants were either sown directly and raised in the same containers or they were first raised separately as seedlings and transplanted into the experimental containers a few days before the treatment. The application rate for the postemergence treatment was 31.2 or 15.6 g of a.S./ha.

The plants were kept species-specifically at temperatures from 10-25° C. or 20-35° C. The experimental period extended over 2 to 4 weeks. During this time, the plants were tended, and their reaction to the individual treatments was assessed.

Assessment was carried out on a scale from 0 to 100. 100 here means no emergence of the plants or complete destruction of at least the above-ground parts and 0 means no damage or normal course of growth.

The plants used in the greenhouse experiments were made up of the following species:

| | |
|---|---|
| BIDPI | common blackjack |
| SEFTA | Faber's foxtail |

On postemergence treatment and at an application rate of 31.2 g/ha, compound Ia.3 showed very good action against SETFA and BIDPI (100%) and at an application rate of 15.6 g/ha good to very good action against BIDPI (80%) and very good action against SETFA (100%).

For comparison purposes, the compound of the formula

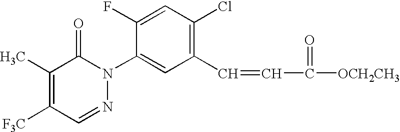

(Example I-658 of WO 97/07104 trans isomer) was investigated. On postemergence treatment and at an application rate of 15.6 or 31.2 g/ha, in the case of BIDPI only a 30% destruction/damage was observed. At an application rate of 15.6 g/ha, the action against SETFA was only 80%.

### II.2 Action as Desiccants/Defoliants

The test plants used were young, 4-leaved (calculated without seed leaves) cotton plants which had been raised under greenhouse conditions (rel. atmospheric humidity 50-70%, day/night temperature 27 or 20° C.).

The young cotton plants were subjected to foliar treatment until dripping wet with an aqueous preparation of the respective active compound, which additionally contained 0.15% by weight, based on the total weight of the preparation, of a fatty alcohol ethoxylate (Plurafac® LF 700). The amount of water applied was approximately 1000 l/ha. After 13 days, the number of shed leaves and the degree of defoliation were determined. The untreated control plants showed no defoliation.

We claim:

1. A 2-phenyl-2H-pyridazin-3-one compound of the general formula I 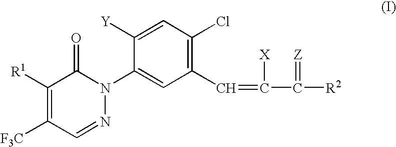
in which the variables R¹, R², X, Y and Z have the following meanings:
X is halogen;
Y is fluorine or chlorine;
Z is oxygen;
R¹ is hydrogen or C₁-C₄-alkyl;
R² is chlorine, OR³ or NR⁴R⁵, in which
R³,R⁴ independently of one another are hydrogen, C₁-C₄-alkyl, C₁-C₄-haloalkyl, hydroxy-C₁-C₄-alkyl, C₁-C₄-alkoxy-C₁-C₄-alkyl, cyano-C₁-C₄-alkyl, C₁-C₄-alkylthio-C₁-C₄-alkyl, C₁-C₄-alkylsulfinyl-C₁-C₄-alkyl, C₁-C₄-alkylsulfonyl-C₁-C₄-alkyl, amino-C₁-C₄-alkyl, C₁-C₄-alkylamino-C₁-C₄-alkyl, di(C₁-C₄-alkyl)amino-C₁-C₄-alkyl, hydroxycarbonyl-C₁-C₄-alkyl, (C₁-C₄-alkoxy)carbonyl-C₁-C₄-alkyl, C₁-C₄-haloalkyloxycarbonyl-C₁-C₄-alkyl, (C₁-C₄-alkylthio)carbonyl-C₁-C₄-alkyl, aminocarbonyl-C₁-C₄-alkyl, (C₁-C₄-alkyl)aminocarbonyl-C₁-C₄-alkyl, di(C₁-C₄-alkyl)aminocarbonyl-C₁-C₄-alkyl, C₁-C₄-haloalkoxy-C₁-C₄-alkyl, C₁-C₄-alkoxy-C₁-C₄-alkoxy-C₁-C₄-alkyl, hydroxycarbonyl-C₁-C₄-alkoxy-C₁-C₄-alkyl, (C₁-C₄-alkoxy)carbonyl-C₁-C₄-alkoxy-C₁-C₄-alkyl, aminocarbonyl-C₁-C₄-alkoxy-C₁-C₄-alkyl, (C₁-C₄-alkyl)aminocarbonyl-C₁-C₄-alkoxy-C₁-C₄-alkyl, di(C₁-C₄-alkyl)aminocarbonyl-C₁-C₄-alkoxy-C₁-C₄-alkyl, C₃-C₆-alkenyl, C₃-C₆-alkynyl, C₃-C₄-alkenyloxy-C₁-C₄-alkyl, C₃-C₄-alkynyloxy-C₁-C₄-alkyl, C₃-C₄-alkenyloxycarbonyl-C₁-C₄-alkyl, C₃-C₄-alkynyloxycarbonyl-C₁-C₄-alkyl, C₃-C₆-cycloalkyl, C₃-C₈-cycloalkyl-C₁-C₄-alkyl, C₃-C₈-cycloalkoxy-C₁-C₄-alkyl; and
R⁵ is hydrogen, C₁-C₄-alkyl, C₁-C₄-alkoxy, C₃-C₆-alkenyl, C₃-C₄-alkenyloxy, C₃-C₆-alkynyl, C₃-C₄-alkynyloxy, C₃-C₈-cycloalkyl, C₃-C₈-cycloalkyl-C₁-C₄-alkyl, C₃-C₈-cycloalkyl-C₁-C₄-alkoxy;
R⁴ and R⁵, together with the nitrogen atom to which they are bonded, can also be a saturated or unsaturated 3-, 4-, 5-, 6- or 7-membered heterocyclic radical which contains 1 or 2 further heteroatoms, selected from oxygen and sulfur, and/or one or two imino or C₁-C₄-alkylimino groups as ring member(s) and/or one or two substituents selected from halogen, C₁-C₄-alkyl and C₁-C₄-alkoxy;
Z can also be a group NR⁶ if R² is a group OR⁷, in which
R⁶ is C₁-C₄-alkoxy, C₃-C₄-alkenyloxy, C₃-C₄-alkynyloxy, C₃-C₈-cycloalkoxy, C₃-C₈-cycloalkyl-C₁-C₄-alkoxy; and
R⁷ is C₁-C₄-alkyl, C₁-C₄-haloalkyl, C₁-C₄-alkoxy-C₁-C₄-alkyl, cyano-C₁-C₄-alkyl, C₁-C₄-alkylthio-C₁-C₄-alkyl, hydroxycarbonyl-C₁-C₄-alkyl, (C₁-C₄-alkoxy)carbonyl-C₁-C₄-alkyl, aminocarbonyl-C₁-C₄-alkyl, (C₁-C₄-alkyl)aminocarbonyl-C₁-C₄-alkyl, di(C₁-C₄-alkyl)aminocarbonyl-C₁-C₄-alkyl, C₁-C₄-haloalkoxy-C₁-C₄-alkyl or C₁-C₄-haloalkyloxycarbonyl-C₁-C₄-alkyl, C₁-C₄-alkoxy-C₁-C₄-alkoxy-C₁-C₄-alkyl;
or the agriculturally utilizable salts of compounds of the formula I.

2. A compound as claimed in claim 1 of the general formula I, in which R¹ is methyl.

3. A compound as claimed in claim 1 or 2 of the general formula I, in which Z is O and R² is a group OR³.

4. A compound as claimed in one of claims 1 to 3 of the general formula I, in which X is chlorine or bromine.

5. A compound as claimed in one of claims 1 to 4, in which Y is fluorine.

6. A diazinylcinnamic acid compound of the general formula II 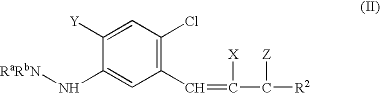
in which
R^{a} and R^{b} are simultaneously hydrogen or form a group ═CH—C(O)—CF₃
and the variables R², R³, X, Y and Z have the meanings mentioned in claim 1.

7. The use of compounds of the general formula I and their agriculturally utilizable salts, as claimed in claim 1, as herbicides or for the desiccation/defoliation of plants.

8. A composition comprising a herbicidally active amount of at least one compound of the formula I or of an agriculturally utilizable salt of I, as claimed in claim 1, and at least one inert liquid and/or solid carrier and, if desired, at least one surface-active substance.

9. A composition for the desiccation and/or defoliation of plants, comprising an amount of at least one compound of the formula I having desiccant and/or defoliant activity or of an agriculturally utilizable salt of I, as claimed in claim 1, and at least one inert liquid and/or solid carrier and, if desired, at least one surface-active substance.

10. A process for controlling undesired vegetation, which comprises allowing a herbicidally active amount of at least one compound of the formula I or of an agriculturally utilizable salt of I, as claimed in claim 1, to act on plants, their habitat or on seed.

11. A process for the desiccation and/or defoliation of plants, which comprises allowing an amount of at least one compound of the formula I having desiccant and/or defoliant activity or of an agriculturally utilizable salt of I, as claimed in claim 1, to act on plants.
